(19) United States
(12) Patent Application Publication
(10) Pub. No.: US 2003/0129749 A1
(43) Pub. Date: Jul. 10, 2003

(54) Detection of immunological memory, T-cell conjugates for pathology imaging and therapy

(76) Inventors: Hans J. G. Gundersen, Horning (DK); Jesper Zeuthen, Hellerup (DK); Steen J.I Nielsen, Hillerod (DK)

Correspondence Address: NIXON & VANDERHYE P.C., 8th Floor, 1100 North Glebe Road, Arlington, VA, 22201, US

(21) Appl. No.: 10/252,112

(22) Filed: Sep. 23, 2002

Related U.S. Application Data
(63) Continuation of application No. PCT/EP01/03250, filed on Mar. 22, 2001.

(30) Foreign Application Priority Data
Mar. 23, 2000 (GB) 0007088.8

(51) Int. Cl. C12N 5/08; G01N 33/567

(52) U.S. Cl. 435/372.3; 435/7.21

(57) ABSTRACT

A method for detecting prior exposure of an individual mammal's immune system to an antigen associated with a pathological process comprises exposing T-cells to a complex antigen mixture, and detecting a pre-existing T-cell specificity for an unknown antigen in said complex antigen mixture. Labelled T-cells are then used to image the site of the pathology and T-cells conjugated to a cytotoxic agent or precursor are used to treat the pathology.

The present invention relates to methods of detecting whether an individual has an immunological memory of an antigen related to a pathological condition or process, in which prior knowledge of the antigen is not required. The invention further relates to methods of producing increased numbers of activated T-lymphocytes responsive to an antigen associated with a pathological process or condition for which an individual has an immunological memory. The invention further relates to methods of determining the position of loci of said pathological process or condition. Lastly, the invention includes novel conjugates of T-lymphocytes conjugated to a material useful in a cytotoxic treatment process.

The mammalian body counters pathological processes via the immune system in a complex manner. As part of this, antigen presenting cells process proteins encountered in the periphery of the body and present peptides derived therefrom bound in the groove of the MHC class I or class II molecules at the cell surface. They migrate to the lymphoid organs where naïve CD8⁺ and CD4⁺ T-lymphocytes bind to the peptide presented in the class I or class II molecules respectively via cell surface receptors and become activated and caused to proliferate to form effector and memory T-lymphocytes of both CD4⁺ and CD8⁺ types.

The proliferated clones of effector and memory T-cells have specific affinity to and specific memory of the antigen peptide. Even after the antigenic stimulus has been removed, some of the T-cells will remain in the circulation bearing that memory, and memory T-cells may be present in the circulation long after the antigen has been removed (Ashton-Rickardt, P. G. et al). Another population of antigen-specific circulating T-cells may, in contrast, be maintained only in the presence of antigen (Sprent J. and Suhr, C. D.) and will disappear from the circulation within a short time if the antigen is removed. These T-cells, which may be antigen-specific effector or memory T-cells, may be characterised by being quickly activated if re-exposed to the antigen in comparison with the activation of long-lived memory T-cells, which typically require a longer period of antigen stimulation.

The presence in the circulation of lymphocytes having memory of a specific antigen associated with a pathological process is evidence that such a pathological process has been experienced by the individual in the past and is ongoing or has been successfully defeated. Specifically, the presence of in vivo antigen-stimulus dependent specific T-cells indicates that the pathological process is or very recently has been present. U.S. Pat. No. 5,601,989 proposes a method of detecting a malignancy in an individual by isolating T-cells from the individual, incubating them with at least one protein expression product of a cancer-related gene associated with the malignancy and detecting the presence or absence of proliferation of the T-cells, it being expected that if there is ongoing malignancy, the antigen presented to the pool of T-cells in the sample will provoke those with a specific memory of that antigen to proliferate.

This method suffers from a number of drawbacks which in practice will severely limit its usefulness. The method presupposes the availability of a purified antigen which is related to the malignancy. Whilst some tumour specific antigens are known, they are relatively few and associated with a small range of the many tumour types that exist (Kawakami, Y. et al).

Whilst it is found that tumours that have grown to detectable size are surrounded and infiltrated by T-cells and therefore must be supposed to be producing antigens to which the immune system can respond, most of those antigens are presently unknown. Even where a specific antigen is known to be produced by a particular type of tumour, there may be other antigens produced by that same tumour which are presently unknown but which may be of great immunological significance. Generally, it is thought that the antigens to which the body responds in producing T-cells that migrate to tumour sites will include the following:

1. embryonic gene products reactivated in the tumour, such as MAGE, BAGE and GAGE family antigens seen in melanoma and a variety of cancers, and Eph A3, CTp11 and CEA.

2. differentiation antigens, such as tyrosinase, MART-1/Melan A, TRP-1,3 and gp 100, all seen in melanoma, and PSA and MUC1.

3. unique or mutated gene products, such as the MUM family antigens, β-Catenin, CDK4, HLA-A2 mutant and Caspase-8.

4. viral gene products, such as those produced by EBV in Burkitt's lymphoma and nasopharyngeal cancer or HPV in cervical cancer;

5. oncogene/suppressor gene products such as survivin. p53, K-ras, HER-2/neu and BCR/abl; and

6. idiotypic epitopes including Ig idiotypes in B-cell lymphoma and TCR idiotypes in T-cell lymphoma.

7. products of genes encoding drug-metabolising enzymes, such as GE2/BF7 involved in metabolism of carcinogens.

8. gene products involved in T-cell activation, such as cyclophilin B.

9. gene products involved in cell division, such as telomerase

Other known and putative human tumour antigens recognised by T-cells are, e.g. GnT-V, p15, PRAME, RAGE, NY-ESO-1/CAG3, LAGE-1/CAMEL, TPI, LDFP, CDC 27, SSX2, SCP-1/HOM-TES-14, CT7, MTG8, GD3, G250, ING1,2, cdr 2, SAGE, RAGE, XAGE-1, F4.2, NA88-A, and SART1 (Kawakami, Y. et al; Wang, R. F.).

The method of U.S. Pat. No. 5,601,989 as exemplified in practice involves incubating the T-cells with a peptide selected from the antigen protein on the basis of its containing a T-cell epitope. However, there can be no guarantee that a particular individual will have an immune response to the peptide selected even if they do have a response to some epitope within that protein.

Although the method described in U.S. Pat. No. 5,601,989 may in a suitable case enable one to determine at a very early stage that an individual is in the process of fighting through its immune system against a cancer producing a specific antigen, the location of the tumour site will remain unknown until the tumour is of a sufficient size to be located via known methods. The prospects of successfully treating a cancer are critically dependant in most cases on early diagnosis, anatomical localisation and treatment, whether by surgical intervention or radiotherapy or chemotherapy or combinations thereof.

U.S. Pat. No. 5,192,537 discloses a therapeutic method which involves taking a patient's own mononuclear cells, depleting them of suppressor cells, and culturing them with an extract of the patient's own tumour and a non-specific lymphocyte activator, preferably with the patient's own serum. The multiplied T-cells so produced, which are activated against the tumour, are reintroduced into the patient to attack the tumour. As the presence of the tumour extract is only optional, it can be seen that the conditions are such that the T-cells are caused to become activated and to proliferate without regard for their antigen specificity.

There are several reports of proliferating T-cells for use in adoptive immunotherapy by cultivating PBMC (peripheral blood mononuclear cells) with lymphokines and autologous tumour cells (Haruta et al; Sporn et al) or with peptides, RNA related to tumour, tumour cell apoptotic bodies (Chang, J. W. et al), recombinant viral oncoprotein pulsed dendritic cells (Santin, A. D. et al, Journal of Virology 1999), or other antigens (Protti et al; Boczkowski et al; Lalvani et al; Plebanski et al; Tanaka et al), but the conditions used in these disclosures are adapted to produce proliferation of T-cells from naive T-cells by primary immunisation. To this end these methods use conditions including the presence of lymphokines, the duration of the process and re-stimulation with antigen that are adapted to achieve proliferation regardless of the presence or absence of T-cells having a previous exposure to the antigen.

T-cells labelled with a radio-label have been used for imaging tumours. For instance, Griffith et al and numerous similar teachings describes labelling tumour infiltrating lymphocytes (TIL) taken from the known tumour of a patient or peripheral blood lymphocytes (PBL) as controls using ¹¹¹In. In several patients, visualisation of tumours was possible using the labelled TIL and in one case using labelled PBL. However, a later report from this group (Pokaj et al) shows that even using TIL there is a high background especially in the lungs, liver and spleen, and that visualisation was only possible outside these areas and in respect of relatively large tumours. In the methods described by these authors, the T-cells are not selected for labelling according to their specificity for any antigen associated with the tumour. TIL may be supposed to contain a higher proportion of T-cells with such specificity than PBL but will also contain other T-cells lacking relevant specificity which will be labelled and which will contribute to non-specific background signal. Furthermore, even the limited specificity of the technique as practised with TIL presupposes that one has already located at least one tumour which can be removed as a source of TIL. The detection of further tumours presupposes that they will display the same antigen that is recognised by a large proportion of the TIL from the removed tumour, whereas it is known that metastases do not always exhibit the same antigen range as primary tumours (Cormier, J. N. et al). A similar problem arises in the imaging method of Mukherji et al. or Santin et al (Santin, A. D. et al, Gynecol. Obstet. Invest. Santin AD et al., Eur J Gynaecol Oncol.), where PBL (peripheral blood lymphocytes) are re-educated by culturing with autologous tumour cells or autologous tumour cell lysate or peptide extracted dendritic cells and IL-2 followed by clonal expansion with IL-2 (and anti-CD3) and subsequent labelling with ¹¹¹In. The cells are re-administered to the patient and imaging of the tumours is achieved. Again, the method is entirely dependent on already having located and biopsied at least one tumour in the patient.

In the case of melamona, Straten et al indicates that each malignant melanoma site within a patient is characterised by its own respective T-cell clonotoypes which do not recirculate to other metastatic lesions and suggests that in situ T-cell responses are heterologous responses, and supports the concept, that T-cells activated in the lymph node migrate to the site of its specificity.

Lymphocytes have also been labelled successfully with ⁵⁵Co for imaging by PET (positron emission tomography), and with ⁵⁷Co for SPECT (single positron emission tomography)—see Korf et al.

To kill pathological cells, it has been widely proposed to conjugate an antibody specific for the cells to a cytotoxic material. This has met with only limited practical success and is limited to cases where it is possible to raise antibodies of the appropriate specificity.

Boron neutron capture therapy has been used in the treatment of tumours. Boron (¹⁰B) containing compounds having a relatively non-specific affinity for tumours have been administered to patients and bombarded locally with thermal (<0.4 eV) or epithermal (0.4-10 keV) neutrons causing fission to produce fast moving ⁷Li and ⁴He particles which cause fatal damage to cells within about one cell diameter of the B atom (Hawthorne; Coderre et al).

It is known that T-lymphocytes activated by encountering an appropriately presented antigen for which they are memory cells are caused to proliferate and that the effector T-lymphocytes so produced migrate to the site of antigen production if it is localised, for instance to tumour sites producing the antigen. Cytotoxic (CD8⁺) T-lymphocytes are better able to home to a tumour site than are circulating antibodies and are better able to penetrate tumours and their cytotoxic effect is highly specific. However, the fact that detectable tumours exist of course demonstrates that the T-lymphocytes are unable to kill all the tumour cells they find at the site. Various methods have been proposed for boosting the cytotoxicity toward tumour cells of effector CD8⁺ T-lymphocytes. These include grafting antibody derived variable regions of desired specificity onto the T-cell receptor constant regions to form so-called T-bodies (Eshhar). In another proposal, bispecific antibodies having affinity for tumour cells but also for the TCR/CD3 complex on CD8+effector cells have been used to redirect the T-cells to target cell-membrane structures of tumour cells (Buen et al).

These methods rely on the cytotoxic ability of the T-cells. All of these methods suffer from the drawback that the tumour must be sufficiently well characterised that it is possible to produce antibodies or antibody regions having the appropriate specificity.

The present invention now provides in a first aspect a method of selectively activating or proliferating one or more T-cell clones each specific for an antigen associated with a pathological process, comprising culturing, under T-cell activating or proliferative conditions, a T-cell mixture potentially including cells having a specificity for at least one said antigen with an effective antigen presenting agent and an antigen mixture, said conditions being sufficiently selective that substantially only T-cells already primed to recognise said antigens are caused to become activated or to proliferate, wherein said antigen mixture has been derived from a microorganism or cell associated with said pathological process by a process comprising lysis, extraction of protein or peptide mixtures, or by the formation of apoptotic bodies, or by being produced in situ from mRNA or DNA derived from said cell or a pathogenic microorganism associated with said pathological process.

The antigen presenting agent may be syngeneic antigen presenting cells (syngeneic with respect to the T-cells) or HLA matched exosomes.

The antigen mixture is preferably derived from a cell associated with said pathological process by a process comprising cell lysis without purification or enrichment of any specific protein or peptide from the cell lysis product. Similarly, where the antigen mixture is produced from apoptotic bodies or by adding mRNA, it is preferred that no particular protein or peptide or mRNA is purified or enriched in the mixture of such materials presented.

As will be seen hereafter, this key method may be used in diagnostic tests for prior immunological exposure to a pathology related antigen without prior knowledge of what the antigen is. It may also be used to detect, isolate and produce large numbers of effector T-cells specific for a pathology related antigen, again without the need for prior knowledge of the antigen itself. Cells so produced may be labelled and used for determining the spatial position of pathological lesions or may be conjugated to material useful in a cytotoxic treatment of the pathology.

In preparing said antigen mixture, after cell lysis, cell membrane debris is preferably removed with the aim of minimising T-cell activation in response to non-self HLA markers on the antigen producing cells. However, this will not in all cases be necessary. Also, purification to improve the effective antigen response following presentation may be considered. In particular, immune suppressive factors may be removed or blocked.

The process of cell lysis can be carried out in a number of known ways, but freeze/thaw cycling of the cells is preferred.

The cell from which said antigen mixture is derived is preferably allogeneic with respect to said T-cells. This will generally be the case in the diagnostic tests described below, but where the eventual aim is to label the T-cells and use them for finding the location of pathological lesions such as tumours, there may be occasions where the cells are from the individual mammal in question (syngeneic or autologous). For instance, if one tumour has been located and removed or biopsied, it may be desired to multiply T-cells having specificity for any antigen produced by the tumour that the immune system has taken note of and acquired memory for. Cells from the tumour would then be used as the antigen source in the method described above. The T-cells produced might then be labelled for the purpose of finding other tumours in the mammal or might be armed with a cytotoxicity related material and used in chemotherapy of the mammal.

Said antigen presenting cells may be at least predominantly dendritic cells. For this purpose, a blood sample from the mammal may be treated in a known manner to cause dendritic cell precursor cells such as monocytes to mature into dendritic cells. Suitable conditions for this are reviewed in Peters et al and are further discussed in Gluckman et al. During the maturation process, T-cells from the sample may be stored, allowing any activated T-cells present to lose their state of activation. The stored T-cells (or T-cells from a further sample from the mammal) may then be added to the matured dendritic cells and cultured in the presence of the antigen mixture.

Dendritic cells take up proteins and peptides from their environment and process them so as to display peptide fragments thereof in both MHC class I and MHC class II molecules on their surface. The ability to display both class I and class II molecules and thus to activate both CD8⁺ and CD4⁺ T-cells is thought to be unique to dendritic cells. Accordingly, this manner of working the invention will produce a multiplied T-cell culture which contains CD4⁺ or CD8⁺ T-cells or both.

An alternative procedure is to use antigen presenting cells which are at least predominantly monocytes. Monocytes are present in blood in much greater numbers than mature dendritic cells, so the step of maturing the monocytes may be omitted. Generally, peripheral blood mononuclear cells (PBMC) containing both monocytes and T-lymphocytes may be used without separating monocytes and T-lymocytes. If desired, already activated T-cells in the sample can be killed by treatment with a suitable antibody such as anti-TAC or can be removed, such as by binding to an antibody on magnetic beads. In the former case, any surplus antibody will itself need to be neutralised before proceeding so that it does not interfere with the activation process. Alternatively, a blood sample taken some time before may have been stored to deactivate any spontaneously activated T-cells before they are used in the method of the invention.

Monocytes are only able to display antigenic peptides in MHC class II molecules and so will only activate CD4⁺ T-cells.

The use of dendritic cells and monocytes will have respective advantages and disadvantages. The avoidance of the step of maturing dendritic cells would be a major advantage from the point of view of speed and lack of complexity in routine use. However, CD8⁺ cells may be needed or advantageous for some purposes, as discussed further below.

The antigen presenting agent may comprise exosomes. These are vesicles secreted by antigen presenting cells including B-lymphocytes and dendritic cells. They are further described in WO 00/28001, WO 97/05900, Thery, C. et al and Zitvogel, L. et al. These membrane vesicles display functional MHC class I and class II T-cell costimulatory molecules. For use as antigen presenting agents in this invention they should be HLA matched with the T-cells of the patient but need not be derived from the patient. The T-cells are preferably obtained from a blood sample (PBL-peripheral blood lymphocytes). Other suitable body fluids may be used as sources of T-cells, including cerebrospinal fluid, bone marrow, pleural effusions, cells within the lymphatic system (lymph, lymph nodes, spleen), peritoneal effusions, urine and sputum in certain cases as well as saliva or tears.

To increase the numbers of antigen-specific T-cells in the sample, individuals may be treated with certain compounds including such that

a) increase the numbers and reactivity of circulating antigen-specific T-cells using cytokines, preferably IL-2 (Demir, G. et al), and IL-12 (Mortarini, R. et al), but possible also INFα, which may induce antigen-specific T-cells (Schmittel, A. et al) or IL-18 enhancing T-cell response (Ju D W et al.).

b) increase the numbers and reactivity of circulating antigen-specific T-cells immunising the patient using vaccines or adjuvants, e.g. BCG etc.

c) increase the immunogenicity of the pathological process, including cytotoxic drugs (Schmittel, A. et al, low-dose whole-body radiation (Cameron, R. B. et al; & Safwat, A.); and IFNγ, which may up-regulate tumour-associated antigen expression (Shiloni, E. et al).

d) increase the T-cell co-stimulatory or decrease the T-cell inhibitory effect of cells or substances in peripheral blood and/or around the pathological process, including compounds that increase the numbers or function of circulating antigen presenting cells such as GM-CSF, IL-4 (Roth, M. D. et al), and Flt3 ligand (Morse, M. A. et al).

Said antigen mixture may derive from a cancer cell. Antigen mixtures from more than one type of cell may be combined or cells of different types may be mixed before or after lysis. Said antigen mixture may derive from other kinds of cells involved in a pathological process including cells infected by a parasite, fungus, bacterium, virus, or prion. Parasites include protozoa and amobae. Suitable examples of such cells will include cells from patients infected by tuberculosis, malaria, leprosy, HIV, aspergillus, cytomegalovirus or prion diseases such as Creutzfeld-Jacob disease. Pathological processes producing chronic, encapsulated localised lesions are of particular interest.

The microorganism may be a bacterium or virus and may be lysed to provide the antigen mixture by known methods. Viruses may be lysed by detergent.

T-cells having pre-existing specificity for an antigen (as opposed to naive T-cells) may be considered to be categorisable as (1) memory cells and (2) in vivo activated effector T-cells. Effector T-cells are or have very recently been exposed to antigen, normally when fighting a pathology. The memory T-cells may be divided into those which are resting and those which are activated (Sallusto, F. et al). These various T-cells may be caused to show activation signals in in vitro assays and to proliferate under conditions of differing selectivity. Thus, effector T-cells and activated memory T-cells may be easier to activate in vitro than resting memory cells. The selectivity of the conditions used may be employed as a tool to differentiate between present, recent and long ago exposure to antigen. In the case of present exposure through on-going pathology, the extent of activation achieved may be used quantitatively to asses the extent of the pathology. This may be used to determine the probable extent of tumour or other pathology and over a period to measure response to treatment. This includes in relation to malignant disease detecting occult metastases or recurrent malignant disease. Thus, the invention includes such a method practised to determine whether the said mammal is subject to said pathogenic process, for use in order to establish the diagnosis of the pathology, to evaluate the effect of treatment of the process, to estimate whether there may be residuals of the pathological process after treatment, to predict the likelihood that treatment may have an effect on the pathological process or its residuals, to predict the prognosis witn respect to recurrence or final outcome from the process, and to estimate whether the process has recurred after prior treatment.

Further T-cells specifically activated by tumour-cell lysate may be isolated e.g. from PBMC by FACS or using magnetic beads and used to identify T-cell activating tumour or other pathology associated antigens (by assessing the ability of suspected antigens to specifically re-stimulate these T-cells) or to verify the result of the pathology location method described below by testing the reaction of the isolated T-cells to known tumour-associated antigens or to a preparation containing unknown tumour-associated antigens.

In a further aspect, the invention includes a method for detecting prior exposure of an individual mammal's immune system to an antigen associated with a pathological process, comprising obtaining a sample from a mammal, said sample containing T-cells, exposing said T-cells to a library of antigens forming a complex antigen mixture, and detecting a pre-existing T-cell specificity for an unknown antigen in said complex antigen mixture.

As further described below, such a method may detect said specificity by attempting to selectively activate or proliferate one or more T-cell clones each specific for an antigen associated with a pathological process, comprising culturing, under T-cell activating or proliferative conditions, a T-cell mixture from said sample, potentially including T-cells having a pre-existing specificity for at least one said antigen, with an effective antigen presenting agent and a said antigen mixture, said antigen mixture being derived from a microorganism or cell of a type associated with said pathological process by a process comprising lysis, extraction of proteins or a peptide mixture, or by the formation of apoptopic bodies, or being produced in situ from mRNA or DNA derived trom said cell or pathogenic microorganism associated with said pathological process.

Also however, such a method may comprise exposing said T-cells to a capture agent comprising said antigens of said library so as to bind to said capture agent T-cells having a pre-existing specificity for an antigen in said library. Said library of antigens may comprise peptides bound to MHC molecules. The MHC molecules may be in the form of multimers (which term is to include dimers and tetramers) and may be bound to a carrier such as a magnetic bead.

Methods of binding T-cells having a pre-existing specificity for a peptide are disclosed in WO96/26962, WO99/13095 and in Luxembourg et al. In these previous methods however, the specificity of the T-cells was essentially known and a single peptide was used rather than a complex mixture of unknown peptides, including peptides from a multitude of different proteins.

However, the methods of presenting peptides bound in MHC class I or MHC class II molecules described in these publications can be adapted for use in the present invention. Complex peptide mixtures may be contacted with the MHC molecules to allow binding between the MHC molecules and such peptides in the mixture as can bind to the MHC molecules, and the resulting bound peptides can be presented to the T-cells of the sample to see which if any will bind to the T-cell receptors.

Thus, T-cells are detected on the basis of their specificity rather than a combination of their specificity and their ability to be activated.

The MHC molecules may be bound in turn to a detectable label of any kind, and may be bound to a solid support which is separable from the mixture, for instance magnetic beads.

In a further aspect, the invention provides a method for detecting prior exposure of an individual mammal's immune system to an antigen associated with a pathological process, comprising obtaining a sample from a mammal, said sample containing memory or effector T-cells, attempting to selectively activate or proliferate one or more T-cell clones each specific for an antigen associated with a pathological process, comprising culturing, under T-cell activating or proliferative conditions, a T-cell mixture from said sample, potentially including memory T-cells specific for at least one said antigen, with an effective antigen presenting agent (which may be syngeneic antigen presenting cells or HLA matched exosomes as above) and an antigen mixture, said antigen mixture being derived from a microorganism or cell of a type associated with said pathological process by a process comprising lysis, extraction of protein or peptide mixtures or by the formation of apoptotic bodies, or by being produced in situ from mRNA or DNA derived from said cell or a pathogenic microorganism associated with said pathological process, and detecting said selective activation or proliferation.

Once again, the antigen mixture is preferably not purified to boost the concentration of a preselected antigen.

It will be appreciated that in these diagnostic methods it is not necessary that the patient be known or even individually suspected to have any particular pathological condition. Such a method may be employed when said mammal is asymptomatic with respect to said pathological process. Alternatively, the method may be employed advantageously where some symptoms have appeared which are not diagnostic of the pathological process.

Additionally, the methods, and in particular the method by which in vivo antigen-stimulus dependent specific T-cells are detected, may be employed in mammals when the nature of the pathological process is known, to estimate the extent of pathology, to evaluate the effect or treatment of the process, to estimate whether there may be residuals of the pathologic process after treatment, to detect the presence of metastases of tumours, to predict the likelihood that treatment may have an effect on the pathological process or its residuals, to predict the prognosis with respect to recurrence or final outcome from the process, and to estimate whether the process has recurred after prior treatment.

It is a particular advantage of this technique that it may be employed where the antigens in said antigen mixture are unknown.

Said sample may contain T-cells representative in antigen recognition capabilities of the whole T-cell population of said mammal. This will generally be the case if the T-cells are obtained from a blood sample and no T-cells are selectively killed or removed.

Optionally, said antigen mixture is derived from multiple cell types associated with respective pathological processes.

Preferably however, said attempted activation or proliferation is repeated using one or more further antigen mixtures each being derived from one or more cell types associated with a or a respective pathological process. The term “repeated’ in this sense includes conducting said replications simultaneously with the first said attempted activation or proliferation or subsequently.

There are a number of established methods for detecting activation or proliferation of T-cells (Romero, P. et al.). These include detecting activation by detecting the expression of cytokines including IL-4, GM-CSF (granulocyte/macrophage colony stimulating factor) TNF-α, IL-2, IL-4 (Schmittel A. et al), expression of Fas ligand (Elsässer-Beile, U. et al), intracellular perforin and granzyme B (Ashton-Rickardt, P. G. et al), IL-10, IL-6 and IFN-γ (interferon γ) by the T-cells either in the medium or on the surface of the cells, inside the cells or by PCR-based detection of cytokine mRNA (Kammula, U.S. et al). Similarly, it is envisaged that detection of chemokine or chemokine receptor mRNA could be detected. With some of these methods, antigen-specific effects on other cell populations, i.e. natural killer (NK) cells and monocytes, may be induced and used advantageously.

Proliferation may also be detected by increased uptake of nucleotide sources such as ³H-thymidine or by the ability of the proliferated cells to lyse antigen producing cells, which can be monitored by radioactive ⁵¹Cr or europium release. Other methods include measuring the rate of IL-2 production, Ca²⁺ flux, or uptake of a dye such as 3-(4,5-dimethylthiazol-2-yl)-2,5-diphenyl-tetrazolium.

The cell from which said antigen mixture is derived will normally be allogeneic with respect to said T-cells. Said antigen presenting cells may be at least predominantly dendritic cells or may be at least predominantly monocytes, as discussed above.

Preferably, the T-cells are PBL but other T-cell sources may be used as discussed above.

The antigen mixture will derive from a cell associated with the pathology with respect to which one wishes to determine the prior exposure or non-exposure of the mammal. Such cells may be any of those discussed above in relation to the first aspect of the invention. Said attempted activation or proliferation is preferably conducted against a panel of antigen mixtures derived from respective cancer cell types, or other suspected pathologies such as cells infected by a bacterium, a virus, or a parasite. The antigen mixtures may be tested in the assay procedure either separately or in admixture. Where they are tested in admixture, one may go on in response to a positive finding to test them separately to determine which cell in the panel gave rise to the positive response in the mixture.

A first screen may be against a panel of tumour cell derived antigen mixtures where each is representative of a class of tumour types and a second screen may be carried out in response to a positive reaction to a member of the first screen panel. The second screening may be against a second panel of tumour cell derived antigen mixtures where each is representative of a sub-type of the tumour type to which said member giving a positive reaction in the first screen belonged. A T-cell response to a lysate having at least some tumour specificity or a certain pattern of response to more than one lysate with at least some tumour specificity may be useful for establishing the type of tumour. This would be especially relevant in individuals diagnosed with disseminated cancer of uncertain type in which the primary tumour cannot be found.

The invention provides in a further aspect, a method of producing labelled T-cells adapted to migrate to the location of an antigen producing cell or cell cluster in a mammal, comprising purifying or selectively multiplying in culture, by a method of the invention described herein, T-cells from said mammal specific for an antigen produced by said antigen producing cell or cell cluster, and conjugating a detectable label to said T-cells.

The terms “conjugating” “conjugation” and “conjugated” in this context embrace all forms of association between a T-cell and a label such that the T-cell and label remain localised together. This includes endocytosis of label into T-cells, covalent or non-covalent bonding of a label or a label containing compound to the exterior of a T-cell and covalent or non-covalent bonding of a label or label containing compound to an internal structure in a T-cell.

Said label may be a radio-label, a fluorescent label, a magnetic resonance contrast agent or an X-ray contrast label. Suitable radio labels include ¹¹¹In, ⁹⁹Tc, ⁵⁵Cr, ⁵⁷Cr, ¹¹⁰In, ⁸⁶Y, ⁷⁶Br, ¹²⁴I, ¹²³I, ¹⁸F, ⁵⁵Co, ⁵¹Fe, ⁶⁶Ga, ⁵¹C, ⁵²Mn, ⁴⁸V, ⁸⁴Rb, ⁵⁶Co, or ⁵⁸Co. ¹²⁴I may be conjugated to T-cells by incorporation in their DNA in the form of ¹²⁴I containing 5-iodo-2′-deoxyuridine, a thymidine analog. One advantage of this is that if the cells divide in vivo, the label is shared with the daughter cells. The label will be retained by the T-cells for as long as they survive.

Magnetic resonance contrast labels such as Gd or Fe will include super paramagnetic nanobeads treated with such that they will be ingested by T-cells, for instance by conjugation to a membrane translocating signal peptide (MTSP) such as HIV-1 tat peptide. (Josephson et al; Lewin et al, Dodd et al). As these are negative contrast agents, they may best be visualised using modes of MRI imaging favouring such agents, e.g. TELEX (Sussman M. S. et al), which highlights short T-2 values whilst supressing long T-2 values.

Although the MTSP nanoparticles have been disclosed previously as magnetic resonance contrast agents, the ability of T-cells to ingest such particles may also be exploited using other types of label. Thus, a tracer radio label may be incorporated in such MTSP nanoparticles, either in the core or in a coating (suitably dextran) surrounding the core. The tracer may constitute the core or may be mixed with other core material such as iron oxide.

Essentially all suitable tracer isotopes emit a gamma-quantum (may be via a positron) well above 50-100 keV, and they do not emit beta-particles (at least not of low energy). Examples of low energy tracers are ⁹⁶Tc (140 keV) and ¹³³Xe (80 keV). Cytotoxic isotopes on the other hand, which for use as labels should be avoided, are preferentially alfa-emitters, providing a 1-to-1 kill. High intensity beta-emitters, including low energy electrons (auger) may also be used for cytotoxic purposes (³¹¹I and ¹²⁵I are good examples). Such cytotoxic isotopes may also have a gamma emission but this does not make them suitable as tracer labels.

Fluorescent labels may be used where the tumour location may be probed using light to stimulate fluorescence either applied to the skin or, e.g. applied via intravasal optical fibres.

Preferred radio labels have a half life of a few days or longer, e.g. about three days. Particularly preferred labels include ⁵²Mn, ⁴⁸V, ¹²⁴I, ⁸⁴Rb, ⁵⁶Co and ⁵⁸Co.

T-cells labelled according to this procedure may be administered to the mammal from which they derive and will naturally home to the site of cells producing the antigen for which they have specificity. This may be used to locate a primary tumour or its metastases. Both the tumour type and the presence of the tumour may be previously unknown. By locating such a tumour at a stage in its growth which is earlier than is possible by known methods, one may in many cases allow the surgical removal, radiation therapy, or chemotherapy destruction of the tumour before it has spread to other sites, so that the tumour may be eradicated.

The antigen-specific accumulation of T-cells at the site of pathology is indicative of the specific antigen being present at that site. Thus, the technique is a biological tumour marker and may therefore complement other visualising techniques such as CT, MR, SPECT and PET which are not diagnostic of tumour cells.

Treatment of the individual to enhance the pathology localisation of labelled antigen-specific T-cells may include those that

a) increase the supply of T-cells to the pathological process, e.g. increase the numbers and reactivity of circulating antigen-specific T-cells preimmunising the cancer patient using cancer cell lysate (Mitchell M S et al.) or cancer cell lysate loaded dendritic cells (Nestle F O et al.).

b) increase the immunogenicity of the pathological process,

c) increase the tendency for lymphocytes to adhere to vascular endothelium of the pathological process,

d) increase the tendency of lymphocytes to migrate across the endothelial lining into the pathological process, or

e) decrease the effect or production of inhibitory substances or suppressor factors/cells

In clinical trials, successful localisation of educated peripheral blood lymphocytes in cancer patients has been achieved without adjuvant treatment (Mukherji, B. et al). However, to assist in maintaining the viability of the administered T-cells and to improve their localisation to the tumour, appropriate doses would preferably be given to the patient of one or more lymphokines, preferably IL-2 (Fisher, B. et al; Pockaj, B. A. et al,) and IL-12 (Mortarini, R. et al).

Localisation may possibly be enhanced by administration of compounds which may increase the numbers and/or function of antigen presenting cells at the tumour site(s) such as GM-CSF and IL-4 (Roth, M. D. et al,; Kim, J. A.) and Flt3 ligand (Morse, M. A. et al.

Also, the expanded T-cell culture could be treated to facilitate T-cell localisation through induction with chemokines or induce the expression of chemokine receptors, integrins, L-selectin and other surface proteins facilitating homing of the expanded T-cells (Agace W W et al)

The administration of a cytotoxic agent may be preferred as one clinical study showed increased localisation of TIL in tumours of cyclophosphamide pretreated patients (Pockaj, B. A. et al), possibly caused by depleting endogenous suppressor cells or exposing antigen binding sites. To this end low-dose total-body irradiation might be considered also (Safwat, A.). Direct or mediated transduction of tumour cells with cytokine encoding genes may enhance tumour immunogenicity (Rosenberg, S. A.).

Intra-venous administration of labelled T-cells is preferred. However, in some cases alternative routes such as intra-arterial or intra-thecal infusion, or infusion into pleural, peritoneal or other body cavities, into lymphatic vessels, or peri- or intra-lesional infusions may increase the supply of T-cells to relevant anatomical sites.

The present invention provides in a further aspect a method of determining the location of an antigen producing cell or cell cluster in a mammal, comprising administering said labelled T-cells to the mammal that was the original source of said T-cells, allowing said T-cells to migrate to the location of said antigen producing cell or cell cluster, and detecting the location of said migrated T-cells from said label.

In a further aspect, the invention provides T-cells specific for an antigen, said T-cells being conjugated to a cytotoxic material, or to a material capable of being transformed in vivo into a cytotoxic material, or capable of causing a pro-form of a cytotoxic material to be transformed in the local vicinity of the T-cell into said cytotoxic material. The term “conjugated” has the meaning given above. The term “cytotoxic” should be understood here in a broad sense to include all factors that will lead to the stasis or death of pathological cells or microorganisms via mechanisms other than the killing activity of the T-cells themselves. These mechanisms will include angiogenesis inhibition, growth reducing factors, differentiation inducing factors and immunomodulatating factors. Mechanisms that lead to a conversion of pathological cells into a non-pathological form are included. However, factors that merely improve the natural action of T-cells by for instance promoting their homing to tumour sites or redirecting their specificity to target a tumour protein are excluded. Thus any material capable of exerting a localised therapeutic effect independent of the cell killing activity of the T-cells themselves is included.

The activated, expanded T-cells can not only be modified for therapeutic purposes with radionuclides and cytotoxic substances, but also can be genetically modified for therapeutic purposes. These modifications could include genes involved in affecting cells for improved immunological recognition, factors inhibiting cell growth/division, factors inducing apoptosis, factors affecting cells supporting the pathology, e.g. in cancer affecting endothelial cells lining the blood vessels using e.g. inhibitors of VEGF (vascular endothelial growth factor)(Claudio P P et al.; Ding I et al.), or the T-cells could be infected with viruses producing the above mentioned substances.

Preferably, the T-cells are conjugated to a potentially cytotoxic material capable of being transformed into cytotoxic form in the body of a mammal by localised administration of a stimulus to the body of the mammal.

By way of example, the potentially cytotoxic material may be ¹⁰B which is transformable in vivo by bombardment with thermal neutrons to produce a particles and ⁷Li. Boron may be conjugated to T-cells by methods described below.

More specific details of methodology that may be employed in the various aspects of the invention will now be described.

Methods for isolating PBC from blood samples are widely described in the literature (U.S. Pat. No. 5,858,358; Tanaka et al). Generally, PBC may be isolated by leukophoresis followed by density gradient centrifugation, using for instance a Ficoll-Hypaque gradient.

Dendritic cells are not common in the peripheral blood but may be obtained from the same mammal by a process of maturing and differentiating monocytes as described in the literature (Peters et al; Gluckman et al) by culturing monocytes with GM-CSF, IL-4 (interleukin-4) and optionally TNF-α (tumour necrosis factor α) over a period of a few days.

Monocytes may be isolated from the peripheral blood of the same mammal by their property of adhering to plastic surfaces. Thus, after mononuclear cells are separated from other blood components by Ficoll/Hypaque centrifugation, monocytes may be captured and separated from lymphocytes by adhering the monocytes to a plastic surface simply by suspending the cells in a suitable medium in a plastic tissue culture flask and decanting non-adherent T-cells. However, generally the most suitable method for obtaining monocytes for use in antigen presenting cells is simply to irradiate PBMC so as to prevent the T-lymphocytes therein from proliferating whilst leaving the monocytes fully functional. Monocytes for use in re-stimulating T-cell clones so as to propagate them may be prepared this way, but for presenting antigen to T-cells in a diagnostic assay it will not generally be necessary to separate the monocytes and PBMC may be used as obtained from the patient.

For use in presenting antigen to primed T-cells, dendritic cells or monocytes may be allowed to adhere to the walls of a suitable vessel such as a multi-well assay plate or purified using anti-CD14 coated magnetic beads. The antigen mixture may be added to such cells before or with the T-cells which are to be tested or proliferated. If there has been a significant lapse of time between the isolation of the T-cells and their use, they may be stored by freezing in a known manner.

During an immune response to a peptide displayed by an antigen presenting cell in its MHC I or MHC II T-cells expressing a T-cell receptor having a high affinity for the displayed peptide in its MHC context become activated and are induced to proliferate. In the first encounter with such a displayed peptide, small numbers of T-cells will proliferate to form memory T-cells and effector T-cells specific for the antigen peptide. After the immune challenge has ended, the effector T-cells will undergo apoptosis but a population of memory T-cells will remain. If the challenge is ongoing, both memory T-cells and effector T-cells will be present in the blood sample taken (Ashton-Rickardt, P. G. et al).

Subsequent encounter with the same antigen by the memory T-cells or effector T-cells if present will lead to a faster and more intense response either in vivo or in vitro. The strength of the response in vitro may be measured in accordance with the invention by measuring the degree of activation or proliferation of the T-cell population upon re-exposure to the antigen. Previous studies suggest that memory T-cells fall into two broad categories on the basis of their activation status (Sallusto, F. et al). Typical memory cells, which may be termed “resting” memory cells, are relatively quiescent and need to be re-activated before expressing effector function. A second category of memory cells displays many of the features of effector cells and may be termed “activated” memory cells. Activated memory cells may represent a subset of cells that retain T-cell receptor contact with small quantities of specific antigen (Sprent, J. et al). Thus, activated memory T-cells may be dependent on very recent contact with antigen in vivo, and may, with respect to malignancies, therefore act as an indirect tumour marker. The detection of activated memory T-cells rather than resting memory T-cells may be based on the fact that the first-mentioned T-cells already to some extent are activated and therefore secrete cytokines after only a very brief contact with antigen in vitro (typically in the range of few hours). Thus, wheras activated memory cells have been shown to be capable of secreting IFN-γ within 6 hours of antigen contact (Lalvani, A. et al), resting memory T-cells, in contrast, would require longer antigen contact to be activated (typically many hours to days).

The existence of and the possibility to detect such in vivo antigen-contact-dependent T-cells in cancer patients was supported by our data (see Example 14). Activation of antigen specific T-cells produces a complex series of changes in the T-cell which may be used to detect and measure the process. Activation events include cross-linking of certain cell surface molecules, intra-cellular events leading to the production of certain enzymes, increased mRNA and protein synthesis including production of certain lympho/monokines and activation antigens, expression of certain activation antigens including lympho/monokine receptors on the cell surface, replication of DNA and cell division. Herein we refer to the relevant events leading up to cell division as ‘activation’ and to cell division itself as ‘proliferation’, but it will be understood that proliferation is itself a consequence and therefore a measure of activation.

Dendritic cells and monocytes are capable of causing proliferation of naive T-cells to produce expanded T-cell populations specific for an antigen which has not previously given rise to a cellular immune response. This not only occurs in vivo but is readily achieved in vitro. A number of disclosures of methods relying on this are discussed above. However, when practising the present invention, the conditions should be chosen such that such primary immunisation of T-cells by antigen presenting cells does not occur to a significant extent. This is easily achieved by limiting the duration of the activation of the T cells by the antigen mixture to less than 48 hours, preferably not more than 24 hours, e.g. about 16 hours, and by not re-stimulating the T-cells with antigen after a period of days as is done in primary immunisation methods. The aim according to the invention is to expand pre-existing antigen specific T-cells in the sample. The detection of activated T-cells using assays detecting cytokine-producing cells may also detect non-T-cells, such as macrophages and NK-cells increasing the number of antigen-specific cells.

Suitable assays for activation include measurement of cell surface expression of cytokines like IL-4 or IFN-γ. This may be detected by the filter immunoplaque assay, otherwise called the enzyme-linked immunospot assay (ELISPOT) (Romero, P. et al). Assays may be designed to label the activated T-cells with a detectable label or with a label enabling the T-cells to be isolated by targeting cell surface bound IL-4 or IFN-γ with a suitable antibody. Commercial assay kits for this purpose are available for instance from Miltenyi Biotec (Gladbach Germany). Cells producing as few as 100 molecules of a specific protein per second can be detected. Also the detection of activated lymphocytes could be performed using multiparameter flow cytometry. The ELISPOT assay uses two high-affinity cytokine specific antibodies directed against different epitopes of the same cytokine molecule. The assay normally involves coating a cytokine-specific antibody to a nitrocellulose-backed microtitre plate, blocking the plate to prevent non-specific absorption, incubating the cytokine-secreting cells at several different dilutions, adding a labelled second anti-cytokine antibody, and detecting the antibody-cytokine complex.

Serial dilutions of the T-cells under test are added to the plate wells. The stimulus to T-cell activation may be provided either prior to the assay or during the incubation of the T-cells on the plate. Incubation of the cells with antigen stimulation on the plate typically takes 6 to 24 hours in a humidified 37° C., 5% CO₂ incubator. Several primary and secondary antibodies to suitable cytokines are commercially available.

Secondary antibody is added and incubated for 2 hours followed by alkaline phosphatase-labelled detecting antibody (e.g. avidin, streptavidin, or goat anti-rabbit IgG) and further incubation for 1 to 2 hours. BCIP/NBT is added to each well and incubated to form blue spots as a positive reaction. Alternatively, horseradish peroxidase conjugated protein may be used as a detecting agent with aminoethyl carbazole (AEC) as a substrate, producing brown spots. Also, the ELISPOT might be used to measure the secretion of other proteins produced by activated T-cells, e.g., granzymes or perforin could be detected in addition or alone.

The activation and culturing of activated lymphocytes could be facilitated using various co-stimulatory factors. This could include anti-CD28 (Sansom D M et al) enhancing the T-cell response, various cytokines such as IL-2, IL-4, IL-7 or IL-15 sustaining cell survival (Vella A. T. et al)

Once an antigen mixture which produces specific activation of the patient's T-cells has been identified, the procedure may be repeated preparatively to produce large numbers of T-cells of the required specificity by prolongation of the activation process. Generally, this may be achieved by continued culture of the specific T-cells with periodic restimulation with IL-2 and/or other cytokines, like IL-4 (Lewko, W. M. et al) and/or growth factors and/or chemokines until the required numbers of T-cells are obtained. Limited contact with further antigen mixture during such continued culture may be desirable, but care should be taken to maintain the conditions such that naive T-cells are not immunised against the antigen to any substantial extent.

In this process, and optionally also in the original screening for memory against the antigen mixture, one may select a sub-population of the T-cells on which to work. For instance, IL-10 positive T-cells may be removed by antibody capture as IL-10 may be suppressive of the desired activation and clonal expansion. This could be done using MACS select marking IL-10 secreting cells with IL-10 using a T-cell-surface antigen—IL-10 bispecific antibody and subsequently removing IL-10 marked cells using IL-10 specific magnetic beads. Also subpopulations of T-cells, e.g. CD4+ or CD8+ cells could be preselected for analysis using anti-CD4+ or anti-CD8+ labelled magnetic beads.

CD4⁺ or CD8⁺ T-cells, may be preferentially selected using antibody coated magnetic beads. Also antigen specific cells might be selected by way of specificity. Using for example magnetic beads coated with tetrameric complexes loaded with peptides, antigen specific T-cells could be magnetically labelled for purification (Dunbar P. R. et al). The peptides could be obtained, e.g. through degradation of cancer cell lysates. Preferably the peptide loaded tetrameric complexes should be HLA-matched.

The expanded T-cell clones may be treated to enhance their survival in vivo so as to improve localisation to a site of pathology. This may be by carefully adjusting the culturing conditions to in vivo conditions or by culturing in the presence of one or more agents enhancing survival or by genetic engineering of the T-cells to produce such an agent. This may be by transfection with a suitable vector containing DNA encoding the agent, e.g. adenovirus (Hirschowitz, E. A. et al), or a retroviral (Willemsen, R. A. et al) or lentiviral (Costello, E. et al) vector, or a gene-containing plasmid (Schmidt-Wolf, I. G. et al).

The agent itself may be an apoptosis inhibitor such as survivin (WO98/22589), p53 caspase inhibitors, or agents that induce apoptosis survival genes, such as Bcl-2 or Bcl-xL or reduce apoptosis inducing genes such as Bax or Bcl-xS or other factors, e.g. transforming effector T-cells into memory T-cells or factors specifically regulating the survival, proliferation, and differentiation of T-cells, corresponding to the B-cell effector molecules like BAFF and APRIL (Laabi Y and Strasser A.).

T-cells may be labelled for tumour or other antigen producing pathology localisation by radio-labelling with ¹¹¹In by incubation with say 500-1000 μCi per 10¹⁰ cells ¹¹¹In oxine for 15 minutes with gentle rocking in PBS as described in Fisher et al. T-cells may be labelled by uptake of [¹⁸F] fluordeoxyglucose or by ⁹⁹Tcm hexamethyl-propylene amine oxime (HMPAO) (Botti C et al).

IgG can be labelled with ⁹⁹Tc (Mishra et al) using ⁹⁹Tcm pertechnetate after treatment of the IgG with stannous chloride dihydrate, ascorbic acid and GHA. Thus, T-cells can be labelled with radiolabelled IgG directed against a T-cell surface protein inessential for the bio-functionality of the cells. Antibodies used for in vivo administration preferably should be human or humanised. Alternatively, antibodies can be made less immunogenic by PEGylation.

T-cells may be labelled by endocytosis of a suitable label. Josephson L. et al discloses labelling T-cells with magnetic resonance contrast agents comprising superparamagnetic iron oxide nanoparticles which are coated with a crosslinked aminated dextran and derivatised with a membrane translocation signal peptide (MTSP). Several such translocation signals have been described (Lewin et al) but the preferred one is an HIV-TAT peptide. This results in the incorporation of very substantial quantities of iron into each cell, of the order of 10¹³ atoms/cell. This method may be used for labelling lymphocytes according to the invention for magnetic resonance imaging. However, by replacing the iron oxide core of the nanoparticles with a radio nucleotide, large quantities of radio-label may be incorporated into T-cells. Alternatively, by binding a radio-label to the nanoparticles before or after endocytosis, large quantities of radio-label may also be incorporated into each cell.

PET (positron emission tomography) and SPECT (single positron emission computed tomography) labels are preferred for use in the invention. SPECT labels include 123I, ¹³¹I and ⁵¹Cr. PET labels include ⁵²Mn, ⁴⁸V, ⁸⁴Rb, ⁵⁶Co, ⁵⁸Co, ¹¹⁰In, ⁸⁶Y, ⁷⁶Br, ¹²⁴I, ¹⁸F, ⁵⁵Co, ⁵²Fe and ⁶⁶Ga. Cells may be labelled with metal chelates. Suitable labels include Co(II) oxine, Co(III) tropolonate, Fe(III) oxine, Ga(III) oxine and Ga(III) MPO.

T-cells may readily be labelled with radioactive iodine, including ¹²⁴I, by proliferation in the presence of 5-[¹²⁴I] iodo-2′-deoxyuridine which becomes stably integrated into the DNA as an analogue of thymidine. Preparation of the reagent and cell labelling methods are described in Guenther et al. The half life (4.15 days) and ready incorporation into T-cells make ¹²⁴I a preferred PET label. ¹²³I and ¹³¹I may similarly be used as SPECT labels.

As described in Korf et al, lymphocytes may be labelled with Co simply by incubation with CoCl₂ which is taken up by the cells in a similar manner to calcium.

Labels may be incorporated in microbeads or liposomes and attached to T-cells by antibody linking or endocytosed by T-cells For attachment, antibody to cell surface antigens of T-cells may be used where the antigens are chosen such that the antibody binding will not interfere with the viability of the T-cells or their ability to home to tumour or other pathology sites. β2 microglobulin or CD45 represent suitable sites for antibody specificity.

One type of target for localisation according to this aspect of the invention is local lymph nodes containing metastases (sentinel nodes).

For use in neutron capture radiotherapy to kill off tumour cells or infected cells, T-cells may be labelled with ¹⁰B, which has an extremely large cross section for neutron capture. One suitable method involves the coupling of boron rich oligophosphates to sulphydryl groups introduced into the CH2 domain of a chimeric IgG (Guan et al). Another method involves reacting the lysine residues of IgG with m-maleimidobenzoyl succinimide ester followed by Michael addition to the maleimido group by the mercapto boron cage of mecaptoun-deahydro-closo-dodecaborate (Ranadive et al). Suitably labelled IgG may be produced for binding T-cells. Boron compounds may be incorporated into micro-capsules, micro-beads, or liposomes and bound to T-cells by antibody linkage. Boronated porphyrins, nucleosides, nucleotides and other boronated compounds may be ingested by T-cells.

Large quantities of boron may be endocytosed by T-cells if presented as boron compound nanoparticles analogous to the iron oxide nanoparticles of Josephson L. et al. Colloidal forms of boron are available as described in Celik M. S. et al. They may be stabilised by coating with dextran as per Joesphson L. et al and my be derivatised with any suitable membrane translocation signal, such as a TAT peptide. Other therapeutic materials than boron may be endocytosed in the same way. These may be other neutron capture agents as described herein or they may be radio nuclides as described below. Loads of over 10 pg B per CTL, e.g. about 20 pg/CTL, may be achieved. Boron may be incorporated in a coating (e.g. of dextran) over a core of other material such as iron oxide instead of the boron being placed in or constituting the core.

As described in Sano, a boron enriched streptavidin may be produced and this may be conjugated to T-cells which have been suitably biotinylated. Biotinylation may be accomplished by modification of the method described in relation to B lymphocytes in Jakob et al. Once T-cells labelled with ¹⁰B have been injected into the patient and allowed to migrate to the pathology site, the site is exposed to thermal neutrons. Desirably, one will achieve a concentration of at least 40 ppm boron in tumour tissue, with preferably a concentration ratio of better than 1:3.5 between normal tissue and turnover tissue. This may be achieved if one T-cell containing say 10^{13 10}B atoms in ingested beads localises to each 10,000 cancer cells.

Other agents proposed for use in neutron capture therapy may also be used, these include ¹⁵⁷Gd and also ³He, ⁶Li, ¹¹³Cd, ¹⁴⁹Sm, ¹⁵Eu, ¹³⁵Xe, ¹⁵⁵Gd, ¹⁶⁴Dy, ¹⁶⁸Yb, ¹⁸⁴OS, ¹⁷⁴Hf, ²³U, ²⁴¹Pu, ²⁴²Am, ¹⁹⁶Hg and ¹⁹⁹Hg.

As the T-cells are adjacent or surrounding the neutron capture agent it is to be expected that they will be killed immediately when the pathology site is irradiated with thermal neutrons. However, repeat neutron irradiation may still be possible at intervals over a period without the administration of fresh T-cells depending on the rate of clearance of the neutron capture agent from the site. This is expected to be low.

Other cytotoxic agents may be conjugated to T-cells in a similar manner.

These may be cytotoxic radionuclides, or ¹³¹IU dR.

Other preferred isotopes for radiotherapy are those which fulfil certain criteria, i.e.:

1. half-life between 1 and 10 days

2. chemically reactive (for binding to a carrier)

3. mostly beta-emitting (>90% of the energy)

4. gamma energies relatively high, 120 to 350 keV (for external monitoring)

5. stable or longlived (low emission) daughter nuclei.

Ideal according to these criteria are

³²P, ³⁵S, ⁷⁷As, ⁹⁰Y, ¹¹¹Ag, ¹⁴⁹Pm, ¹⁶¹Tb and ¹⁷⁷LuAlso suitable, but less preferred are

⁴⁶Sc, ⁶⁷Cu, ^{80m}Br, ⁸⁹Sr, ¹⁰⁰Pd, 125I, ¹²⁹Ba, ¹³³I, ¹⁴⁰La, ¹⁵³Sm, ¹⁶⁵Dy and ¹⁹⁸Au.

In order to increase uptake of the T-cells by the tumour the site of administration may be adjusted to bring the T-cells into early contact with the appropriate location.

Similar treatment of patients as described for enhancing localisation of antigen-specific T-cells to sites of pathology could be used to increase the chance of specific carrier-bound T-cells locating to sites of pathology. Additionally, prior to T-cell harvest, the numbers of antigen-specific T-cells in vivo could be increased by treating patients as mentioned previously in association with the test for antigen-specific T-cell activation. Also, prior immunisation could increase the numbers of antigen-specific T-cells in circulation (Kammula, U.S. et al).

In addition, treatment directed against anatomic sites with known pathology could enhance the homing of T-cells at these sites. This treatment might include localised external irradiation (Santin A. D., et al, Gynecol. Oncol. 1996, 60: 468-474), local infusion of cytotoxic agents, local injection of lymphokines stimulating T-cell function such as IL-2 or IL-12, or local administration of compounds which may increase the numbers and/or function of antigen presenting cells at the tumour site such as GM-CSF, IL-4 (Roth, M. D. et al), Flt3 ligand (Morse, M. A. et al), and a number of other cytokines (Baggers, J. et al).

Injection of T-cells into the arterial system or tumour supplying vessels, into or around tumours or into cavities such as the peritoneal cavity may increase the number of lymphocytes accumulating around the tumour as was shown in animals (Basse, P. H., APMIS 1995; S55: 5-28).

Other therapeutic agents may be conjugated to T-cells in modifications of currently proposed methods in which antibodies are conjugated to therapeutics. Thus, low toxicity pro-drugs may be transformed in situ into powerful cytotoxic agents by a suitable enzyme. The enzyme may be delivered to the pathology site by conjugation to T-cells or expression by genetically modified T-cells. Alternatively, the pro-drug may be delivered by conjugation to T-cells and may be activated by enzyme delivered separately. One method would be to conjugate the enzyme or have it expressed on or secreted by T-cells which are separately administered to home to the pathology site. An alternative would be to administer an enzyme antibody conjugate, where the antibody is targeted either to the pathology or to the T-cells bearing the pro-drug.

Many examples of pro-drugs and co-operating enzymes are known in this art, including by way of example cephalosporin based pro-drugs and lactamase enzymes or ifosfamide, a cytochrome. Membrane-binding of drugs may be used. By example Zy-Linkers are membrane-binding lipophilic dyes which can be-incorporated into lymphocytes and conjugated to various therapeutic agents including doxorubicin (Goldfarb, R. H. et al).

T-cells may be genetically altered to produce other herapeutic agents e.g. by transfection with a vector encoding the therapeutic agent. This may, as described previously, be through the use of an adenovirus vector (Hirschowitz, E. A. et al), or a retroviral (Willemsen, R. A. et al) or lentiviral (Costello, E. et al) vector, or a gene-containing plasmid (Schmidt-Wolf, I. G. et al). The therapeutic agent produced by genetic engeneering may, e.g. be a cytokine, such as IL-2 (Schmidt-Wolf, I. G. et al), or TNF-α (Hwu, P. and Rosenberg, S. A.) or IL-12 (Hirschowitz, E. A. et al), a chimeric antibody/T-cell receptor directed against for example ErbB-2 and TCR, respectively (Altenschmidt, U. et al), a herpex simplex virus suicide gene (Niranjan, A. et al), or an inhibitor of a growth factor such as VEGF (Davidoff, M. et al).

As known in the antibody directed chemotherapy art, rescue agents may also be directed to the pathology site to protect normal tissue in the vicinity against damage by chemotherapeutics.

A cytotoxic or cytostatic agent linked to a T-cell may act directly against tumour cells. However, agents are also known that function mainly by enhancing radiation damage in radiotherapy. It will be advantageous if the concentration of such an agent in the tumour area can be boosted relative to that elsewhere in areas of the body that will be exposed to the radiation. Accordingly, such radiation damage enhancers may be linked to T-cells in accordance with the invention. An example is Epirubicin.

In an alternative approach a potent toxin may be bound to an agent that blocks its toxicity. The binding may be broken locally in the tumour by energy transfer from photons within or close to the visible range. Such photochemical processes are conventionally applied in photodynamic treatment, of which there are many known examples. Generally, the limiting factor is the penetration of light through tissue. Light penetration is much better for long wavelengths towards the IR region, whereas the photochemical process is more efficient for shorter wavelengths. The energy transfer is generally larger in multiphoton interactions (two-photon in particular) and at resonnant frequencies, which maximises energy transfer and minimises the damage to healthy tissue. High intensity, short-pulsed laser may provide sufficient penetration for multiphoton energy transfer. Accordingly, such combinations of toxin and toxicity blocking components may be coupled to T-cells according to the invention.

T-cells specific for an antigen and conjugated to a cytotoxic material or other material for use in treatment of the pathology in accordance with this invention may be prepared by a process of activation and clonal expansion using an antigen mixture under conditions selective for stimulating a pre-existing T-cell memory as previously described. This is however not essential and other methods of obtaining antigen specific T-cells having the ability to home to a site of pathology may be used. For instance, T-cells may be stimulated with antigen under less selective conditions such as to immunise the T-cells or re-educate them against the antigen. For this purpose, the antigen may be a single antigen rather than a mixture. It may be a peptide associated with the pathology. Numerous proteins are candidates for use as antigens or as a source from which to derive antigen peptides. These include:

a) proteins encoded by activated oncogenes that may produce, e.g. growth factors, growth factor receptors, mutated signal transducing stations and transcription factors (e.g., ras, myc, EGF-receptor, abl, MDM2, HER2/neu, EGF/c-erB),

b) proteins encoded by mutated suppressor genes like p53, Rb, p16, p19 and APC,

c) proteins encoded by activated or mutated apoptosis regulating genes like p53, survivin, bc1-2, bax and bad,

d) proteins encoded by mutated DNA-repair genes like “mismatch repair genes”, BRCA1 and BRCA2,

e) proteins encoded by activated or mutated genes involved in cellular ageing like telomerase,

f) marker proteins related to the clonal origin of lymphoid malignancies, including idiotype, isotype or clonotype.

g) differentiation marker proteins associated with carcinomas, like proteins being constituents of mucus,

h) differentiation markers associated with tumours of neuroectodermal origin, like melanoma-specific antigens, ganglioside, neural cell adhesion molecules, and tenascin,

i) differentiation marker proteins from the different haemopoietic cell lineages, such as CD10, IL2-receptor, CD5.

j) proteins encoded by activated or mutated genes involved in angiogenesis, e.g. VEGF.

k) proteins encoded by activated or mutated genes involved in immuno-surveillance.

l) proteins being components of the extracellular matrix including the basal membrane, such as laminin (and its corresponding cellular receptor (integrines)), and metalloproteases (e.g., ADAM 12),

m) tissue specific proteins, e.g. tyrosinase, MART-1/MELAN A, TRP-1, and gp 100.

n) oncofetal proteins, e.g. the MAGE family antigens, α-fetoprotein, human chorionic gonadotrophin, placental alkaline phosphatase, and carcinoembryonic antigen.

o) microbiological proteins including viral, fungal, bacterial and prion proteins,

p) proteins involved in or produced during inflammation and tissue destruction.

q) chemokines.

Serving as an example of a relevant peptide from which to derive an antigen, survivin is a protein expressed in very low amounts in normal, non-fetal, tissue but expressed at a much higher level in essentially all tumours. It functions as an apoptosis inhibitor and may be essential for tumour cell survival. T-cells may be stimulated in vitro against survivin (Andersen M. H. et al; Schmitz M. et al).

T-cells cultured and produced in all of these ways may be used to form therapeutic conjugates as described herein.

The various aspects of the invention described herein may of course be used in a combination of any two or more thereof. Typically, the presence of a T-cell memory to a component of an antigen mixture will be found in a first step. The location of an associated pathology will be found in a second step as described and T-cells modified to carry a therapeutic agent to this site will be prepared and administered in a third step.

The invention in its various aspects will be further described and illustrated by the following description of preferred examples. In the following discussion reference is made to the accompanying drawings, in which:

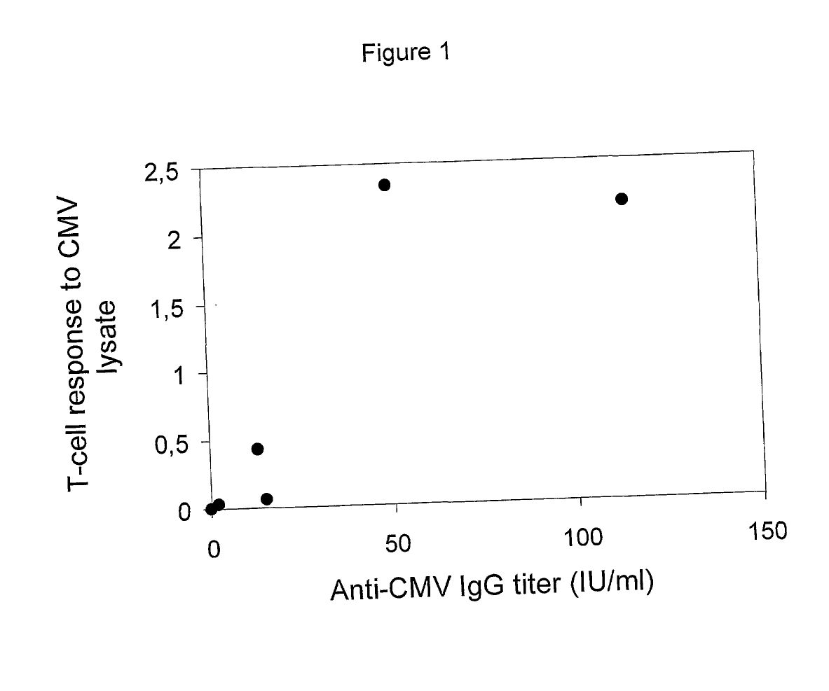

FIG. 1 is a graphical presentation of results derived in Example 13 correlating CMV status assessed by anti-CMV IgG titer by ELISA to T-cell response to CMV lysate by FIFIC;

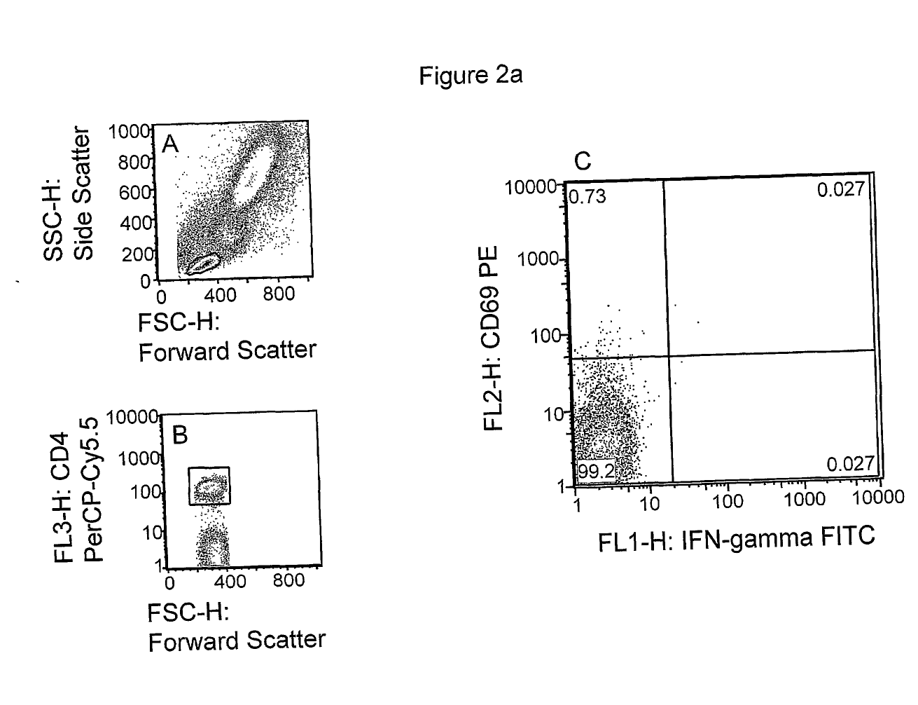

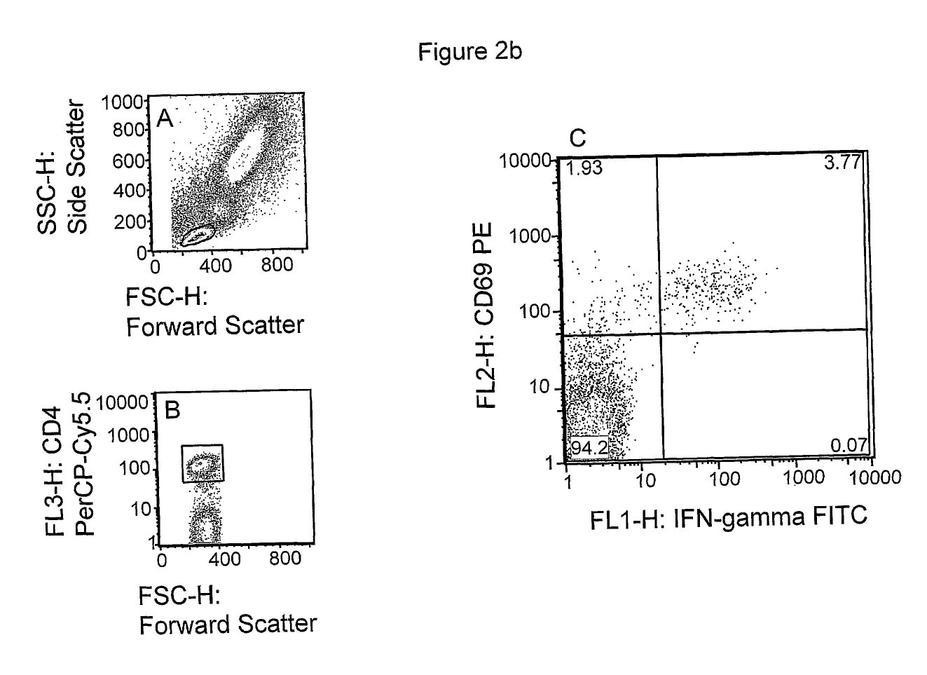

FIGS. 2a and b show results obtained by FIFIC in Example 13;

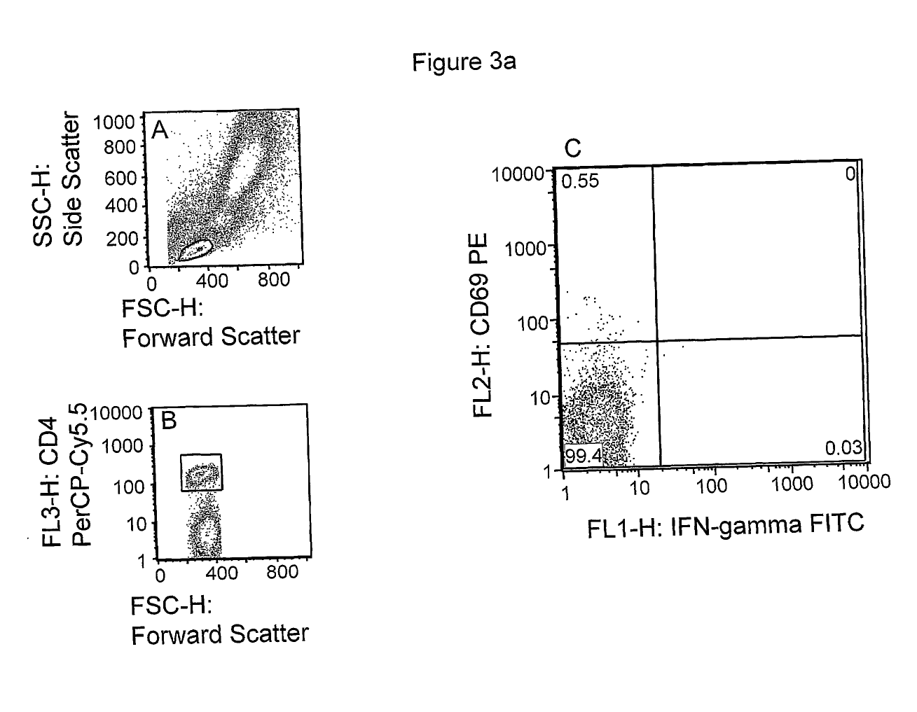

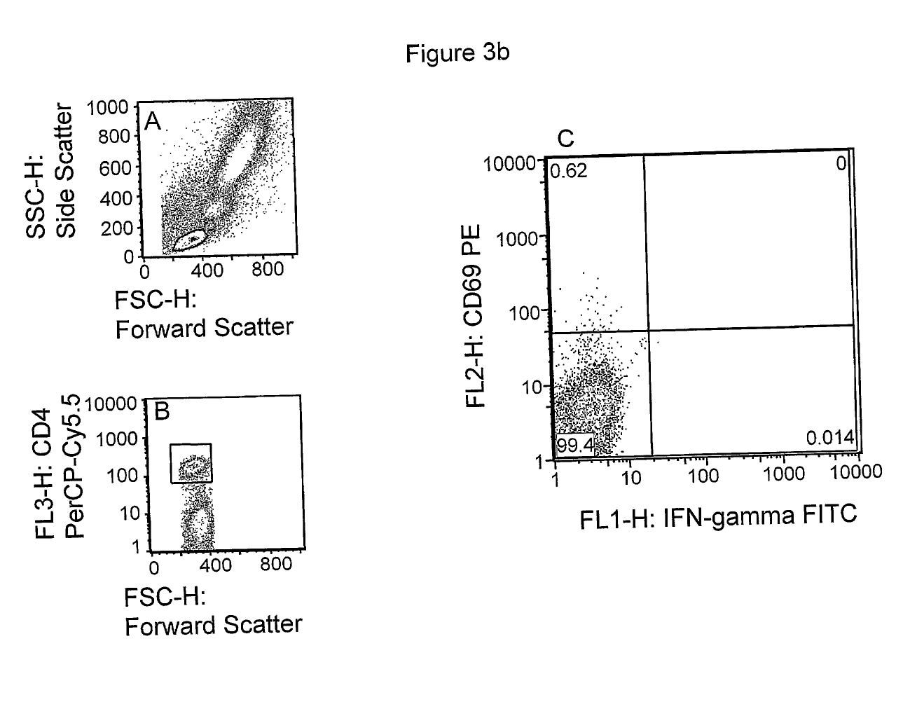

FIGS. 3a and b show results obtained by FIFIC in Example 13;

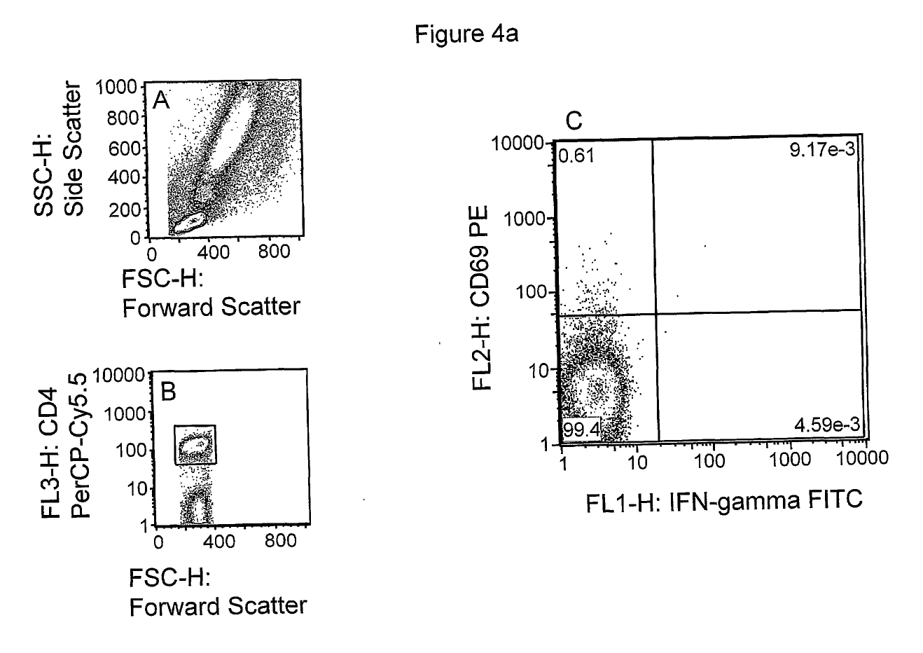

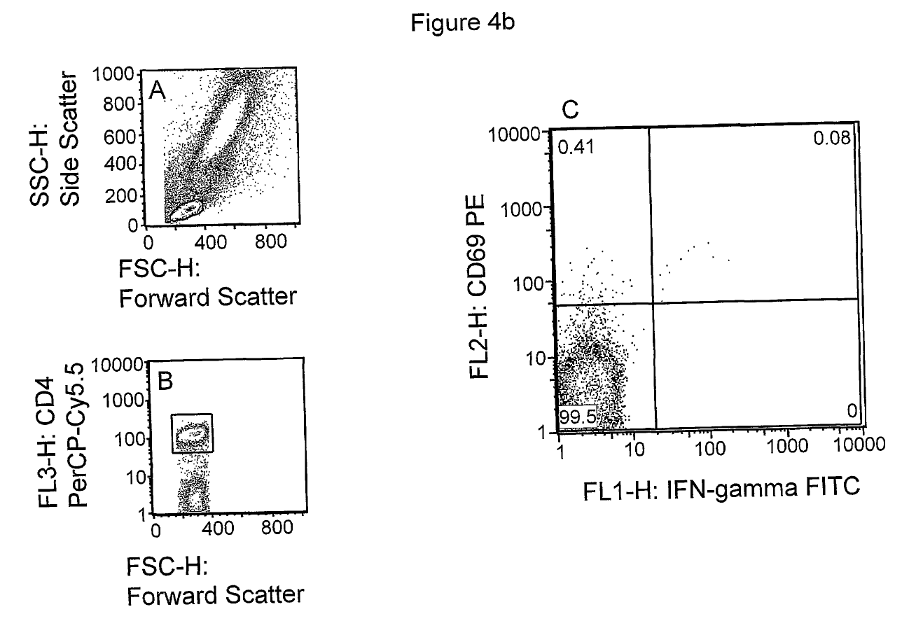

FIGS. 4a and b show results obtained by FIFIC in Example 14;

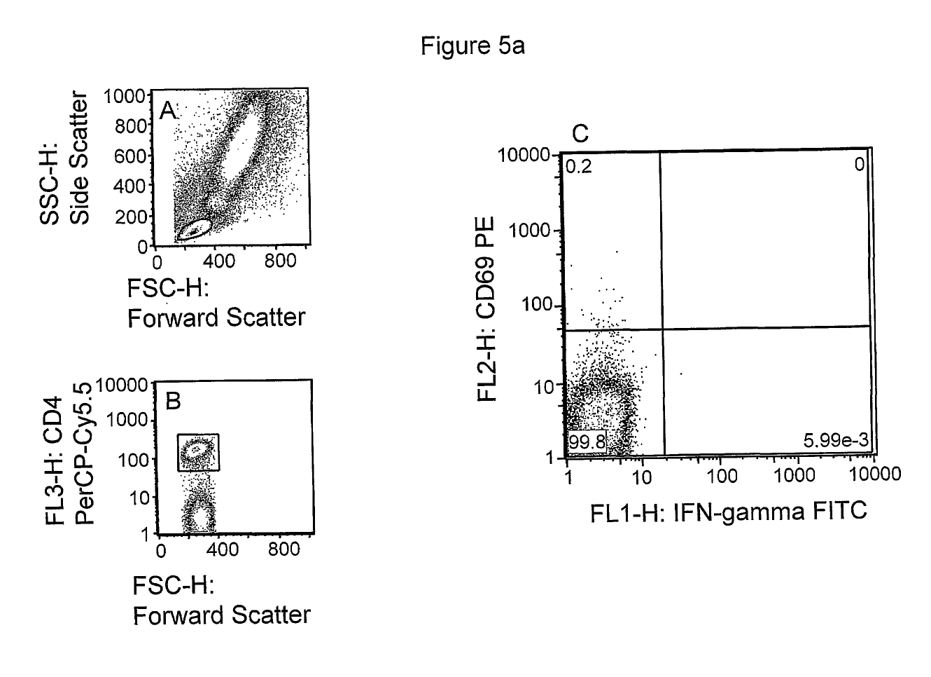

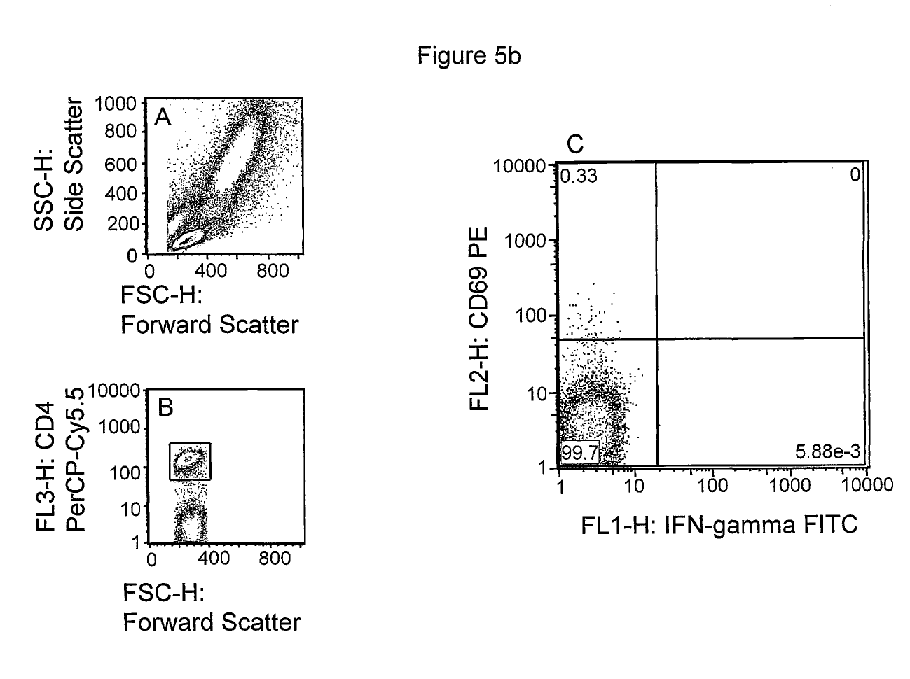

FIGS. 5a and b show results obtained by FIFIC in Example 14;

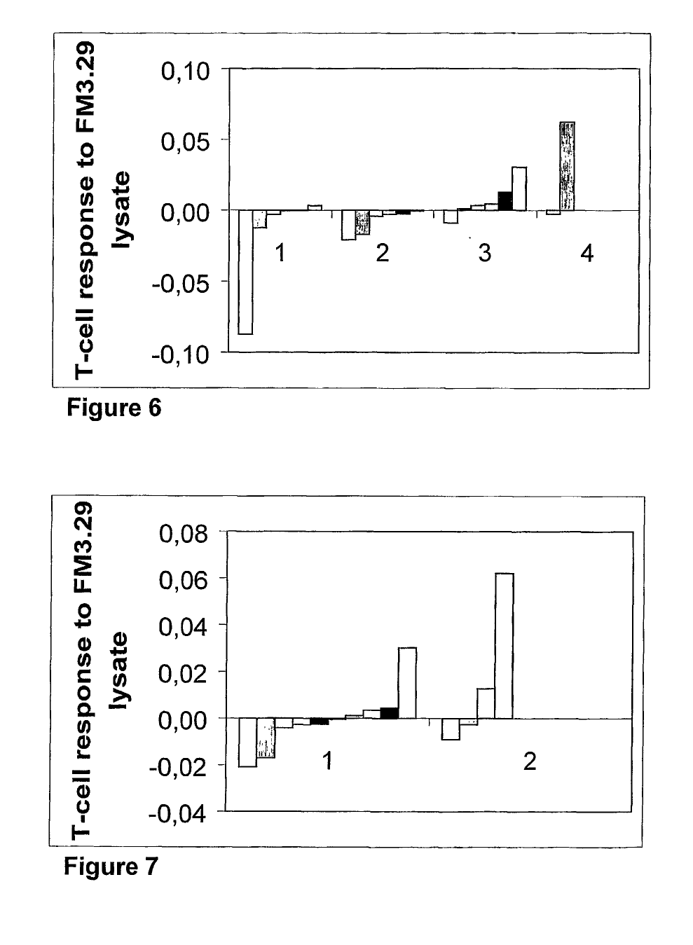

FIG. 6 shows results obtained in Example 14; and

FIG. 7 shows results obtained in Example 14.

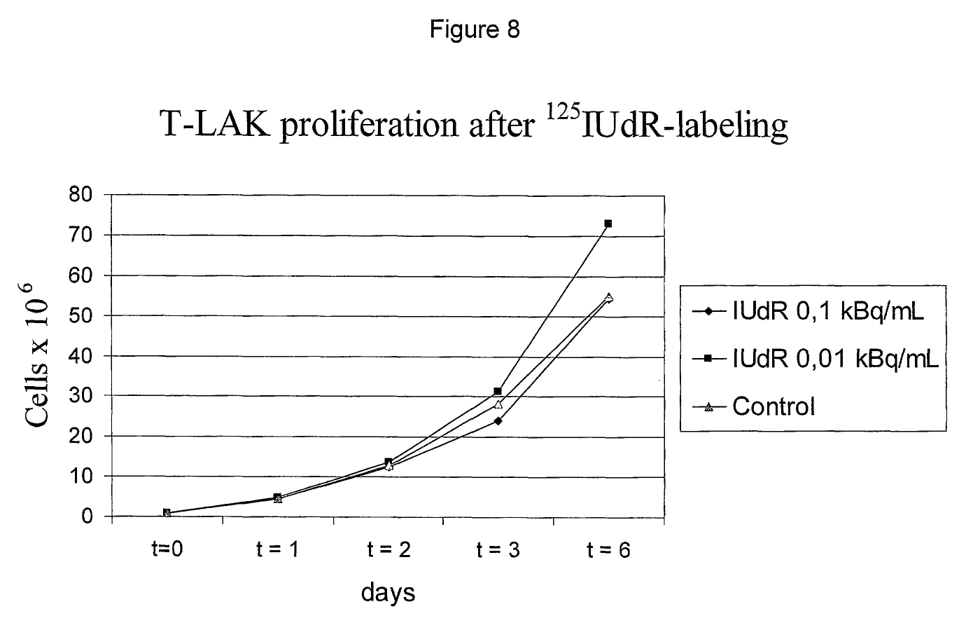

FIG. 8 shows a graph of the proliferation of activated lymphocytes labelled with 0.1 kBq/mL or 0.01 kBq/mL ¹²⁵IudR.

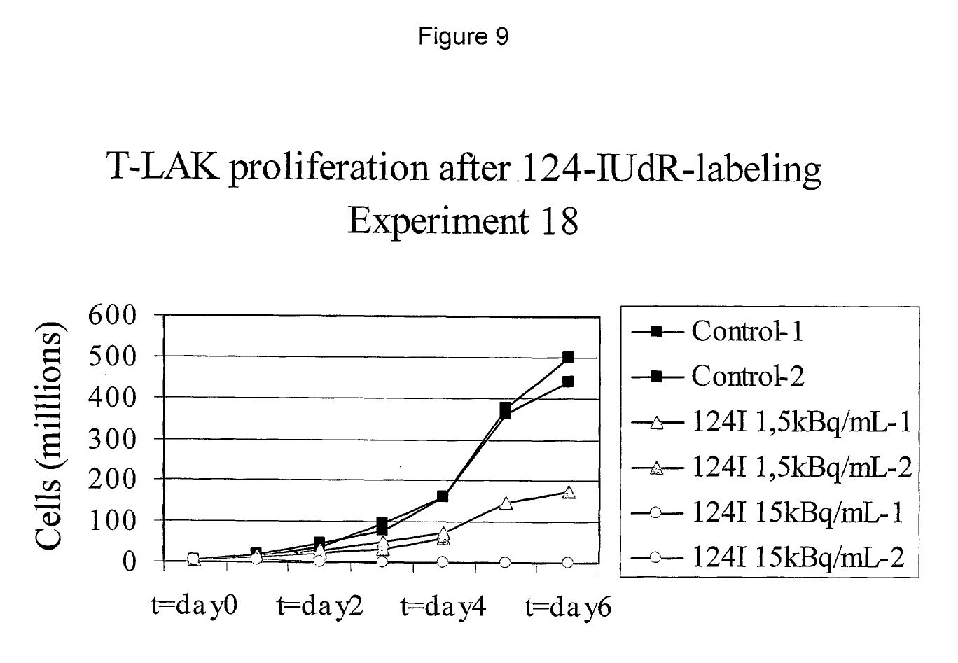

FIG. 9 shows a graph of the proliferation of activated lymphocytes labelled with 1.5 kBq/mL or 15 kBq/mL ¹²⁴IudR.

### EXAMPLE 1

Isolation of T cells from Peripheral Blood

T-lymphocytes are obtained from peripheral blood by Ficoll-Hypaque density gradient centrifugation. Fresh heparinised blood is placed into centrifuge tubes with an equal volume of PBS. Ficoll-Hypaque solution is layered beneath the blood PBS mixture. Centrifugation is carried out for 30 minutes at 2000 rpm (900 g) at 18-20° C. The upper layer containing platelets and plasma is removed and the next layer containing the mononuclear cells is then removed by pipette. The cells are washed by adding excess HBSS, centrifuging for 10 minutes at 1300 rpm and removing the supernatant. The cells are resuspended in HBSS and the washing process is repeated to remove most of the remaining platelets.

The cells may be depleted of monocytes/macrophages by exposure to the plastic surface of a tissue culture flask as follows. The cells are centrifuged for 10 minutes at 1400 rpm, supernatant is removed and the cell pellet is resuspended in complete RPMI-20 to a final concentration of 2×10⁶ cells/ml. The suspension is incubated in the tissue culture flask for 1 hour at 37° C. Nonadherent lymphocytes are decanted into a centrifuge tube and centrifuged for 10 minutes at 1400 rpm. The process is repeated once.

### EXAMPLE 2

Generation of Immature Dendritic Cells

To generate dendritic cells, buffy coat mononuclear cells are isolated on Lymphoprep. 60 ml of buffy coat is diluted to 100 ml with HBSS and layered on 15 ml of Lymphoprep. Centrifugation is carried out in two stages. In the first step centrifugation takes place for 20 min at 200 g after which 20 ml of supernatant is removed. Next, centrifugation is carried out at 380 g for 20 min. Interphase cells are collected and washed with HBSS four to five times at 200 g. Cells are counted and re-suspended in culture medium at 10⁷ cells/ml. The cell culture is placed in T25 flasks for 2 hours and non-adherent T-cells are removed with gentle rinsing with warm culture medium. Culture medium containing GM-CSF and IL-4 is added to final concentrations of 88 and 500 U/ml is added. Fresh medium with lymphokines is added without removal of medium from the flasks at days 3 and 5. At day 6 cells are analysed by FACS for the expression of CD1a and CD83 and for phagocytic activity (with Fluorspheres, Molecular Probes). Typically, the generated dendritic cells are 50-90% CD1a positive and less than 10% CD83 positive, with 25-50% of phagocytic cells.

### EXAMPLE 3

Loading of Dendritic Cells with Tumour Lysate

Tumour lysate is prepared by repeating freezing-thawing of tumour cells. Loading is performed as follows. Dendritic cells are washed, resuspended in AIM-V medium (without serum), and placed in 24 well plates, 10⁶ cells per well in 0.5 ml of medium. 0.5 ml of lysate (prepared in AIM medium and corresponding to approximately 500 μg/ml antigen is added to the dendritic cells. After 4-5 hours, or the next day, TNF-α is added to a final concentration of 10-20 ng/ml and the cells are harvested 24 hours later. By FACS analysis such treatment usually up regulates the levels of CD83 expression by up to 60%.

### EXAMPLE 4

Exposure of T-Lymphocytes to Tumour Lysate Loaded Dendritic Cells for Clonal Expansion and ELISPOT Interferon γ Assay for Enumeration of CTL Precursors and Frequencies

Mabtech coating antibody (1-D1K, 1 mg/ml) is diluted to 7.5 μg/ml in sterile PBS and is added at 75 μl/well to a 96-well nirocellulose plate (Millipore, MAIP N45). The plates are left overnight at room temperature and washed with PBS (6×200 μl/well). The wells are blocked with R10, 200 μl/well, being left for 2 hours at 37° C., 5W CO₂ in an incubator.

Serial dilutions of T-cells from Example 1 are made for adding to the wells of the 96 well nitrocellulose plates in 100 μl R10 (dialysed FCS). Tumour lysate loaded dendritic cells are added at 10³-10⁴ per well and incubated overnight at 37° C. without stirring.

The following day, media is discarded and the wells are washed (six times) with PBS containing 0.05% Tween (PBS/Tw) before the addition of biotinylated secondary antibody (7-B6-1-Biotin, Mabtech) at 0.5 μg/ml in 75 μl PBS containing 1% BSA and 0.02% NaN₃ (PBS/BSA). The antibody is added to each well (75 μl/well) and the plate is incubated for 2 hours. The wells are washed with PBS containing 0.05% Tween (6×200 μl/well).

Alkaline phospatase-avidin enzyme conjugate stock (Calbiochem, 189732) is prepared by dilution as prescribed in 2 ml ddH₂O and mixing with 2 ml of glycerol (85%). The product is stored at 4° C. and diluted before use 1:1000 in PBS, 1% BSA, 0.02% NaN₃, and added at 75 μg/well.

After incubation for 1 hour at room temperature, the plate is washed with PBS containing 0.05% Tween (6×200 μl/well).

Fresh substrate is mixed (44 μl NBT (75 mg/ml) and 33 μl BCIP (50 mg/ml)(Gibco cat. 18280-016) with 10 ml substrate buffer (0.1M NaCl, 0.1 M TrisHCl, 50 nM MgCl2, pH 9.5).

The wells are washed once with substrate buffer immediately before use and 75 μl/well of NBT/BCIP mixture is added.

After leaving for 5-20 min covered and monitoring for the development of spots indicating activated T-cells, the reaction is stopped by the addition of tap water.

### EXAMPLE 5

Exposure of T-Lymphocytes to Antigen in the Presence of Monocytes

PBMC containing monocytes are stimulated in vitro with tumour lysates. Briefly, 20×10³ PBMC are incubated with approximately 500 μg/ml antigen protein in 10 ml 15% autologous serum in an ELISPOT assay setup as described previously and spots indicating activation of T-cells by the presented antigen are counted.

### EXAMPLE 6

Proliferation of T-Cell Clones Specific for a Pathology Associated Antigen

PBMC containing monocytes are again stimulated in vitro with tumour lysate derived from a tumour to which a positive response was obtained in Example 5. Briefly, 20×10³ PBMC are incubated with approximately 500 μg/ml antigen protein in 10 ml 15% autologous serum. Activated T-cells are propagated by addition of 20 U/ml Il-2 and are periodically re-stimulated as required with antigen until the desired numbers of T-cells are obtained.

### EXAMPLE 7

Proliferation of T-Cell Clones Specific for a Pathology Associated Antigen Using Dendritic Cells as APC

T-cells are added to the wells of the 96 well nitrocellulose plates in 100 μl R10 (dialysed FCS). Tumour lysate loaded dendritic cells from Example 3 are added at 1M/ml and incubated overnight at 37° C. without stirring. Incubation is continued for one week followed by restimulation with recombinant 20 u/ml Il-2 and irradiated PBMC until the desired numbers of T-cells are obtained.

### EXAMPLE 8

Labelling of T Cells with ¹¹¹In

Approximately 10¹⁰ T-cells having anti tumour antigen specificity produce in Example 7 are washed twice in 100 ml PBS and resuspended in PBS to a volume of 30 to 60 ml. The T-cells are radio-labelled by incubation with 500-800 μCi of ¹¹¹In oxine for 15 minutes with gentle rocking. The labelled cells are washed twice in autologous plasma, re-suspended in 40 ml normal saline and transferred to a 600 ml plastic transfusion bag. 20 ml 25% human serum albumin, 75,000 U IL-2 and 40 ml normal saline are added to the T-cell suspension for a final volume of 100 ml.

### EXAMPLE 9

Imaging of Tumours with Labelled T Cells

Specific T-cell reactivity to allogenic cancer cell lysate is detected in a blood sample from a patient with suspected but unknown cancer. PBMC are collected from the patient's blood by leukapheresis and specific labelled T-cells are produced as in Example 8. The T-cells are reinfused and imaged by gamma camera scan, providing a precise location of the tumour.

### EXAMPLE 10

Labelling of T-Lymphocytes with ⁵⁷Co

Following the method of Korf et al, 4.5×10⁶ T-lymphocytes of a clone produced and proliferated as in Example 6 or Example 7 are incubated in sterile 10 ml polypropylene tubes for 15 to 60 min at 37° C. in Krebs-Ringer HEPES buffer (pH 7.4) containing 0.15 mM CaCl₂ to which is added about 74 MBq [2 μCi] per tube of ⁵⁷CoCl₂. After centrifugation at 1200 rpm for 20 min the supernatant is decanted and the cells are resuspended in 1 ml buffer.

### EXAMPLE 11

A blood test from a person with increased risk of malignant melanoma contains T-cells specifically reactive with an allogenic melanoma cell lysate. Clinical work-up discloses a small uveal melanoma in the right eye. The patient is operated and the reactive T-cells disappear from the blood. During follow-up the patient's blood is tested at regular intervals for melanoma-reactive T-cells. After a period of follow-up melanoma-reactive T-cells reappear in the blood, but despite an intensive examination no tumours can be found. Tumour-reactive T-cells are isolated from the patient's blood, cultured, labelled and re-infused and disclose a single brain metastasis. The metastasis is technically inoperable, however. Tumour-reactive T-cells are produced in great numbers, loaded with boron, re-infused and neutron irradiation applied to the area of metastasis. Despite apparent erradication of the brain metastasis with only scare-like remains detectable on a post-treatment MR-scan, subsequent infusion of labelled T-cells shows accumulation at the former tumour site suggesting the presence of residual melanoma cells.

### EXAMPLE 12

A patient diagnosed with metastatic melanoma is offered immunotherapy with IL-2. The selection of treatment was in part based on a pre-treatment blood test showing high numbers of melanoma-reactive T-cells, indicating an increased chance of the therapy being effective. During the first courses of IL-2 therapy the numbers of melanoma-reactive T-cells increase indicating an immunological response to treatment. In accordance, the metastases regress. During subsequent courses of IL-2 the numbers of melanoma-reactive T-cells decrease indicating therapy failure and IL-2 treatment is halted. Shortly after the metastases progress.

The following includes a description of materials and methods, and preliminary results of experiments analysing the specific T-cell response of human peripheral blood lymphocytes to in vitro stimulation with lysate, analysed with Flow-cytometric ImmunoFluorescence measurement of Intracellular Cytokines (FIFIC). A CMV lysate was employed to evaluate the feasibility of the technique in infectious diseases, whereas an autologous melanoma cell culture lysate was employed to evaluate the feasibility of the technique in cancer.

T-cell response to cytomegalovirus (CMV) lysate was assessed in healthy persons and correlated with serological antibody titer to CMV in Example 13. T-cell response to autologous melanoma cell lysate from the melanoma cell culture line FM3.29 was evaluated in healthy controls as well as in melanoma patients with highly variable tumour burden in Example 14.

Detection of Antigen-Specific T-Cells in Blood with FIFIC General Description of FIFIC:

The main purpose of the assay is to detect rare, antigen-specific T cells through measurements of the cytokine production induced in these cells when incubated with relevant antigens. See also Nomura et al.

Specifically, a small sample of peripheral blood treated with anti-coagulant (sodium heparin) is stimulated by incubation at 37° C. with a lysate containing a complex mixture of relevant antigens. In addition, a stimulatory signal is provided to the T lymphocytes by addition of activating antibodies directed against cell-surface receptors for co-stimulatory molecules (CD28 and anti-CD49d). Stimulation of T lymphocytes may be improved by the addition of autologous dendritic cells.

As a result of the exposure to antigen, antigen-specific T lymphocytes are activated and respond with production of cytokines. In addition, the treatment may result in the induction of other antigen-specific effects on other cell populations, i.e. natural killer (NK) cells, and monocytes.

Following a period of incubation/stimulation, a cell secretion inhibitor (Brefeldin A) is added to the sample, resulting in intracellular accumulation of the cytokines produced.

After a consecutive incubation of 2-10 hours at 37° C. with Brefeldin A, the sample is treated with EDTA, and red blood cells are disrupted by addition of a lysing solution. The cells are then permeabilised and stained for accumulated intracellular cytokines by means of fluorochrome-conjugated specific antibodies. Finally, the stained cells are fixed by treatment with a 1% formaldehyde solution and stored in the dark at +4° C. until analysis by flow cytometry.

Using a flow cytometer, the size (forward-scatter), granularity (side-scatter) and fluorescence intensity at different wavelengths (multi-colour analysis) is registered for each individual cell.

Specific Description of FIFIC:

Materials:

1. BD FastImmune CD4 Intracellular Cytokine Detection Kit for IFN-γ, Becton Dickinson, cat. no. 340970

2. Antigen lysate (e.g. CMV lysate at 50 μg/ml or tumour cell lysate (prepared at 10×10⁶ cells pr. ml)

3. SEB (Staphylococcal Enterotoxin B), Sigma, cat. no. S4881, 50 μg/ml

4. ddH₂O (double-distilled water)

5. HBSS (Gibco BRL, cat. no. 14175-046)

6. PBS—Dulbecco's, without Ca and Mg (Gibco BRL, cat. no. 14190-086)

7. Washing buffer (0.5% Bovine Serum Albumin+0.1% NaN₃ in PBS)

8. Fixing buffer (1% paraformaldehyde+0.1% NaN₃ in PBS)

9. Fresh blood sample in Sodium heparin glass

10. Na-heparin sample glasses (Becton Dickinson, cat. no. 368480)

11. Rack for Na-heparin sample glasses (Becton Dickinson, cat. no. 364887)

12. Needle with plastic tubing and “butterfly” (Becton Dickinson, cat. no. 367282)

13. 15 ml polypropylene tubes, Greiner cat. no. 188.271

14. 5 ml polystyrene tube for FACScalibur, Becton Dickinson, cat. no. 352054

15. Vortex mixer

16. 37° C. incubator

17. Centrifuge: Beckman-Coulter Allegra™ 6R

18. Flow Cytometer

Procedure:

1. Collect the blood sample in a Na-heparin glass and use within 8 hours. Store sample at room temperature

2. Label 15 ml polypropylene tubes according to the experimental scheme (e.g. mark tubes “no antigen”, “SEB”, “CMV lysate”, “tumour lysate” etc.)

3. To each tube, add 220 μl heparinized blood for each sample to be analysed on the FACS (i.e. if 5 different antibody stainings are to be performed on the “SEB” stimulated sample, add 5×220 μl=1100 μl heparinized blood to the SEB tube at this point).

4. To each tube, add 10 μl anti-CD28/anti-CD49d monoclonal antibody cocktail (from the BD FastImmune kit) pr. ml blood.

5. To tubes labelled “CMV lysate”, add 20 μl CMV lysate solution pr. Ml blood.

6. To tubes labelled “tumour lysate”, add 333 μl tumour cell lysate solution pr. ml blood.

7. To tubes marked “no antigen” add HBSS in a volume equivalent to the lysate added to “lysate” tubes in the experiment to compensate for any dilution-effect of the lysate.

8. Vortex all tubes briefly (max. 2 seconds) and incubate at +37° C., typically for 2 hours

9. Take a vial of ×10 BFA from the freezer, thaw and dilute to ×1 by addition of 9 parts sterile PBS solution (i.e. add 90 μl PBS to a vial containing 10 μl ×10 BFA solution)

10. Following the incubation period, add 20 μl ×1 BFA solution pr. ml blood to each tube.

11. Vortex all tubes briefly (max. 2 seconds) and incubate at +37° C., typically for 4 hours, resulting in a total incubation period of 6 hours.

12. After the second incubation, add 100 μl EDTA solution (from the BD FastImmune kit) pr. ml blood to each tube, vortex all tubes thoroughly (ca. 5 seconds) and incubate tubes at room temperature for 15 minutes.

13. Label an appropriate number of 5 ml FACS tubes according to the experimental scheme.

14. Vortex all tubes with blood for a few seconds, and distribute the samples into the FACS tubes as follows: Tubes marked “CMV lysate”: add 230 μl sample to each FACS tube (corresponds to 200 μl undiluted blood) Tubes marked “tumour lysate”: add 290 μl sample to each FACS tube (corresponds to 200 μl undiluted blood) Tubes marked “no antigen”: add a volume of either 230 μl sample (for CMV experiments) or 290 μl (for tumour cell lysate experiments) to each FACS tube—corresponding to 200 μl undiluted blood.

15. To each tube, add 2.0 ml ×1 FACS lysing solution (from the BD FastImmune kit—diluted from the ×10 stock solution by addition of 9 parts pure H₂O). The solution must be at room temperature before addition.

16. Vortex all tubes briefly, and incubate at room temperature for 10 minutes

17. Add 2.0 ml Washing buffer to each tube and centrifuge 5 minutes at 500×g (1600 RPM in the Beckman-Coulter centrifuge)

18. Discard supernatants and add 0.5 ml ×1 FACS Permeabilizing solution 2 (from the BD FastImmune kit—diluted from the ×10 stock solution by addition of 9 parts pure H₂O). The Permeabilizing solution must be at room temperature.

19. Vortex tubes briefly and incubate at room temperature for 10 minutes.

20. Add 2.0 ml Washing buffer to each tube and centrifuge 5 minutes at 500×9 (1600 RPM in the Beckman-Coulter centrifuge)

21. Discard supernatants and add suitable concentrations of antibodies (when using the BD FastImmune kit, add 20 μl of the pre-mixed antibody cocktails (anti-IFN-γ FITC/anti-CD 69 PE/anti-CD4 PerCP-Cy5.5) to relevant tubes).

22. Vortex all tubes briefly (no more than 2 seconds) and incubate in the dark at room temperature for 30 minutes.

23. Add 2.0 ml Washing buffer to each tube and centrifuge 5 minutes at 500×g (1600 RPM in the Beckman-Coulter centrifuge)

24. Discard supernatants and resuspend cell pellets by addition of 200 μl Fixing buffer to each tube.

25. Vortex all tubes briefly, seal and store in the dark at +4° C. Analyse within 24 hours.

### EXAMPLE 13

Detection of Prior Exposure to Cytomegalovirus (CMV) Using CMV-Lysate

The results of the experiments carried out as described above analysing the specific T-cell response of human peripheral blood lymphocytes upon selective in vitro stimulation with CMV lysate is shown in Table 1 and FIG. 1.

FIG. 1 is a graphical presentation of the correlation between CMV status assessed by anti-CMV IgG titer of ELISA and T-cell response to CMV lysate by FIFIC.

Dots correspond to numbers shown in Table 1, however, for persons analysed with FIFIC several times, the average value is used. The x-axis shows the anti-CMV IgG titer in IU/ml, where titer<4 IU/ml is defined as 0. The y-axis shows T-cell response to CMV lysate, where response is % IFN-γ+, CD69+ among CD4+ lymphocytes upon incubation with CMV lysate with background subtracted (i.e. spontaneous IFN-γ production from the unstimulated sample).

Anti-CMV IgG titer was assessed with standard ELISA prior to the FIFIC test. A total of 6 healthy persons were included. The FIFIC-test was repeated at various time intervals 3 times for two CMV positive individuals and 2 times for another CMV positive.

No T-cell response was found in the two persons with negative anti-CMV IgG titer, whereas all four CMV sero-positive persons showed a T-cell response, seemingly correlating in intensity with the IgG titer.

TABLE 1
Anti-CMV IgG titer from ELISA and FIFIC assay for
IFN-γ production.

| | anti-CMV IgG titer | T-cell response to CMV |
|---|---|---|
| ID | (IU/ml)¹⁾ | lysate²⁾ |
| N1_{CMV} | <4 | 0.00 |
| N2_{CMV} | <4 | 0.00 |
| N3_{CMV} | 13 | 0.54/0.34/0.40 |
| N4_{CMV} | 114 | 3.74/0.62 |
| N5_{CMV} | 15 | 0.06 |
| N6_{CMV} | 50 | 1.98/3.13/1.91 |

Notes:
¹⁾titer <4 IU/ml is defined as negative,
²⁾response is % IFN-γ+, CD69+ among CD4+ lymphocytes upon incubation with CMV lysate with background subtracted (i.e. spontaneous IFN-γ production from the unstimulated sample).

Examples of FIFIC plots for a CMV sero-positive person (N4_{CMV}) and a CMV sero-negative person (N2_{CMV}) are shown in FIGS. 2a and b and FIGS. 3a and b, respectively.

General Description of FIGS. 2-5.

FIFIC analyses may be summarised as shown in FIGS. 2-5. In all these figures a dot corresponds to a cell analysed.

In the inserted figure of the upper left corner of FIGS. 2-5, the x-axis shows FSC-H: Forward scatter (which is light scattered by the cell at n arrow angles with respect to the laser beam—an approximate measure of the cell's size) and the y-axis shows SSC-H: Side Scatter (which is light scattered by the cell at an angle of 90 degrees from the laser beam—a measure of the cell's content of granules) of the total population of cells analysed from the blood sample. The encircled area indicates the cell population selected for further analysis, which is regarded as the lymphocytes.

In the inserted figure of the lower left corner of FIGS. 2-5, the x-axis shows FL3-H: CD4 PerCP-Cy5.5 (which is the intensity of PerCP-Cy5.5 fluorescence and indicative of the amount of anti-CD4 antibody bound by the cell) and the y-axis shows SSC-H: Side Scatter (as defined above) of the lymphocyte population selected. The box encloses the CD4+ subpopulation of lymphocytes selected for further analysis.

In the inserted figure at the right side of FIGS. 2-5, the x-axis shows FL1-H: IFN-γ FITC (which is the intensity of FITC fluorescence and indicative of the amount of anti-IFN-γ antibody bound by the cell) and the y-axis shows FL2-H: CD69 PE (which is the intensity of PE fluorescence and indicative of the amount of anti-CD69 antibody bound by the cell). The cells above the horizontal line are considered to be CD69+ and the cells to the right of the vertical line are considered to be IFN-γ+. The lines are placed based on results of a number of preceding experiments, and are kept constant throughout all further experiments. The numbers shown in the corner of each square indicate the percentage of cells contained in the squares. A responding cell is defined as a cell included in the upper right square of the right figure, which represent IFN-γ+ CD69+ CD4+ lymphocytes.

FIG. 2 shows a summary of the results of FIFIC analysis of a CMV sero-positive person (N4_{CMV}).

FIG. 2a shows the results with no CMV lysate added. 0.027% are IFN-γ+ CD69+ CD4+ lymphocytes.

FIG. 2b shows the results with CMV lysate added. 3.77% are IFN-γ+CD69+ CD4+ lymphocytes.

The specific response of the CD4+ lymphocytes to CMV lysate is estimated by 3.77%−0.027%=3.74%

FIG. 3 shows a summary of the results of FIFIC analysis of a CMV sero-negative person (N2_{CMV}).

FIG. 3a shows the results with no CMV lysate added.

FIG. 3b shows the results with CMV lysate added.

Neither with nor without CMV lysate added, no IFN-+ CD69+ CD4+ lymphocytes are observed, and the response is 0.

FIG. 4 shows a summary of the results of FIFIC analysis of a tumour cell (FM3.29) lysate responding melanoma patient (P4).

FIG. 4a shows the results with no tumour cell lysate added.

FIG. 4b shows the results with tumour cell lysate added.

The response is estimated by 0.08%−0.00917%=0.071%.

FIG. 5 shows a summary of the results of FIFIC analysis of a healthy control (N1) with no response to tumour cell (FM3.29) lysate.

FIG. 5a shows the results with no tumour cell lysate added.

FIG. 5b shows the results with tumour cell lysate added.

Neither with nor without tumour cell lysate added, no IFN-γ+ CD69+ CD4+ lymphocytes are observed, and the response is 0.

Our results show that a specific T-cell response against CMV lysate can be detected in vitro in the peripheral blood of anti-CMV IgG positive individuals. No persons with negative anti-CMV IgG-titer showed a T-cell response. A correlation between the size of the anti-CMV IgG-titer and the numbers of T-cells responding to CMV lysate is suggested.

### EXAMPLE 14

Detection of Prior Exposure to Melanoma Antigens Using Allogeneic Melanoma Cell Lysate

The specific T-cell response of human peripheral blood lymphocytes upon in vitro stimulation with autologous tumour cell lysate was investigated in a pilot study of 14 melanoma patients and 6 healthy controls. Clinical data for melanoma patients are shown in Table 2.

TABLE 2
Clinical data for melanoma patients.

| | Age | | Overt | | Cancer-free interval |
|---|---|---|---|---|---|
| ID | (years) | Stage¹⁾ | disease²⁾ | Sex | days)³⁾ |
| P1 | 70 | III | 0 | F | 58 |
| P2 | 67 | I | 0 | M | 16 |
| P3 | 77 | III | + | M | 0 |
| P4 | 63 | IV | + | M | 0 |
| P5 | 50 | I | 0 | F | 30 |
| P7 | 68 | III | 0 | M | 21 |
| P8 | 72 | III | 0 | F | 54 |
| P10 | 28 | I | 0 | M | 14 |
| P11 | 30 | I | 0 | F | 25 |
| P12 | 38 | IV | + | M | 0 |
| P13 | 62 | I | 0 | F | 28 |
| P14 | 33 | III | + | M | 0 |
| P16 | 57 | III | 0 | F | 63 |
| P17 | 55 | I | 0 | F | 41 |

Notes:
¹⁾Maximal stage until blood sample. Staging performed according to AJCC (AJCC cancer staging manual 5^{th} ed., American Cancer Society, Lippincot-Raven, Philadelphia 1997),
²⁾overt disease indicates the known presence of residual primary tumour or metastases at the time of blood sample,
³⁾the interval from latest apparently radical operation (if any) to blood test.
Patients excluded: P6 excluded due to high dose steroid treatment at the time of blood test. P9 and P15 were not tested with FIFIC.

Patients and Controls:

All patients were in good performance (WHO Performance Status 0-1) and had histologically verified malignant melanoma. They had not been treated with chemotherapy, radiotherapy, hormone therapy or immunotherapy, had not received antihistaminic drugs or blood transfusions within 8 weeks, and had no known autoimmune disease, immunodefect syndrome, or chronic or acute infection. Patients had experienced no other kinds of cancer, except P7 who had a basocellular cutaneous carcinoma removed 3 years before blood test.

A total number of 6 healthy controls (3 males and 3 females) were tested simultaneously with the patients. Healthy controls were selected according to the following criteria: age up to 75 years, WHO Performance Status 0-1, no former malignant or pre-malignant disease, no treatment with antihistamine within 8 weeks, no systemic steroid treatment, blood transfusions, acute or chronic infection, autoimmune disease or immunodefect syndrome.

The study was approved by the local Ethics Committee and Datatilsynet. A written informed consent was received from all persons included.

For patients included, the average age at blood sample was 55 years (range, 33-77 years). 6 patients had or had had stage I disease, 6 patients stage III and 2 patients stage 1V disease. 4 patients had overt disease at the time of blood sample. Male and female patients were equally distributed. The disease-free interval ranged from 0 days to 54 days with an average of 25 days.

Tumour Cell Lysate:

The following describes the procedure used for preparation of lysate from human melanoma cells of cell-line FM3.29:

1. Cells are grown in RPMI 1640 plus 2% pooled human serum in T175 flasks. After reaching 70-90% confluence, medium is changed for RPMI 1640 without serum for 2 days.

2. Harvest cells by trypsinization (0.05% trypsin/EDTA).

For this medium is transferred into 50 ml tube, culture is rinsed with 6 ml of trypsin, trypsin is removed and flask is left at room temperature. Cell detachment could be seen after approximately 30 sec. Add 10-15 ml of removed culture medium, re-suspend cells, and transfer into the same 50-ml centrifuge tube.

3. Wash cells twice in RPMI 1640, resuspend in RPMI 1640, count, and adjust to 10×10⁶/ml. Place into 50 ml tubes, 4-6 ml in each tube.

4. Subject cells to 5 rounds of freezing (in liquid nitrogen for 10-15 min)—thawing (in water bath, 37° C.) Thawing should be done till the moment of ice disappearance, followed by immediate freezing.

5. Sonicate lysate 15 min in ultrasound bath Metason 200.

6. Collect lysate in one tube. Centrifuge at 500 g, 15 min, 4° C.

7. Transfer supernatant into eppendorf tubes. Centrifuge at 13000 g, 60 min, 4° C.

8. Collect supernatant in one tube, filtrate through 0.2 μM filter.

9. Take 0.1ml for determination of protein concentration.

10. Aliquot lysate in cryotubes, 0.5-1.0 ml/tube, store at −70° C.

Results:

Readouts from FIFIC-diagrams are summarised in Table 3 and Table 4. Autofluorescence-corrected data (see legend to Table 3) were used for further analyses. The specific T-cell response was estimated as the difference in percentages of INF-γ+, CD69+ CD4+ lymphocytes observed after incubation with or without melanoma cell lysate. As can be read, responses were often negative, indicating an inhibiting effect of the tumour cell lysate. Serving as a quality control, the blood of one healthy person was usually included in each experiment, run under similar conditions.

TABLE 3
IFNg+ cells among CD69+ CD4+ lymphocytes

| | | Autofluorescent | | Autofluorescent | |
|---|---|---|---|---|---|
| | | events not removed | | events removed | |
| | | | | Spon- | FM3.29 |
| Experi- | | Spontaneous | FM3.29 | taneous | specific |
| ment | Patient | IFNg+ | specific IFNg+ | IFNg+ | IFNg+ |
| | N1 | 0.0000% | 0.0000% | 0.0000% | 0.0000% |
| I-03 | P1 | 0.0189% | 0.0012% | 0.0189% | 0.0012% |
| | N2 | 0.2924% | −0.2715% | 0.0122% | −0.0122% |
| | P2 | 0.1080% | −0.0097% | 0.0973% | −0.0208% |
| I-07 | P3 | 0.0382% | 0.2646% | 0.0000% | 0.0127% |
| | N1 | 0.0000% | 0.0057% | 0.0000% | 0.0000% |
| I-08 | P4 | 0.0090% | 0.0692% | 0.0090% | 0.0621% |
| | N3 | 1.5192% | −1.1147% | 0.1022% | −0.0872% |
| | P5 | 0.1867% | −0.0739% | 0.0312% | −0.0170% |
| I-09 | P6 | 1.3248% | −1.2778% | 0.0257% | −0.0257% |
| | N4 | 0.0055% | 0.0011% | 0.0011% | 0.0033% |
| | P7 | 0.0202% | −0.0026% | 0.0014% | 0.0044% |
| I-10 | P8 | 0.0192% | −0.0057% | 0.0011% | 0.0034% |
| | N6 | 0.0000% | 0.0052% | 0.0000% | 0.0000% |
| | P10 | 0.0103% | −0.0051% | 0.0052% | −0.0025% |
| I-15 | P11 | 0.0042% | −0.0021% | 0.0042% | −0.0042% |
| | N7 | 0.0061% | −0.0030% | 0.0031% | −0.0031% |
| I-16 | P12 | 0.0051% | −0.0051% | 0.0026% | −0.0026% |
| | P13 | 0.0065% | −0.0010% | 0.0065% | −0.0028% |
| | P14 | 0.0180% | −0.0180% | 0.0090% | −0.0090% |
| I-17 | P16 | 0.0410% | 0.0302% | 0.0410% | 0.0302% |
| I-18 | P17 | 0.0055% | −0.0037% | 0.0018% | 0.0000% |

Results of FIFIC analyses of T-cell response of melanoma patients and healthy controls. Spontaneous IFN-γ secretion and estimated specific IFN-γ secretion upon in vitro stimulation with autologous tumour cell lysate.

The first column shows the experiment identification number including those patients (P) and healthy controls (N) shown in column two.

Column three shows the percentage of IFN-γ+ CD69+ cells among CD4+ lymphocytes upon incubation without cell lysate (i.e. the spontaneously IFN-γ producing cells).

Column four shows percentage of IFN-γ+ CD69+ cells among CD4+ lymphocytes upon incubation with FM3.29 tumour cell lysate with the percentage of spontaneously IFN-γ-producing cells subtracted (i.e. the FM3.29-specific IFN-γ producing cells).

Column five and six are similar to column three and four, except that data are corrected by removal of events thought to represent false, “autoflourescent” events* as defined above.

TABLE 4
IFNg+ cells among CD69+ CD4+ lymphocytes

| | | |
|---|---|---|
| | Autofluorescent | Autofluorescent |
| Experi- | events not removed | events removed |

| ment | Patient | FM3.29 | No antigen | FM3.29 | No antigen |
|---|---|---|---|---|---|
| | N1 | 0.0000% | 0.0000% | 0.0000% | 0.0000% |
| I-03 | P1 | 0.0201% | 0.0189% | 0.0201% | 0.0189% |
| | N2 | 0.0209% | 0.2924% | 0.0000% | 0.0122% |
| | P2 | 0.0983% | 0.1080% | 0.0766% | 0.0973% |
| I-07 | P3 | 0.3028% | 0.0382% | 0.0127% | 0.0000% |
| | N1 | 0.0057% | 0.0000% | 0.0000% | 0.0000% |
| I-08 | P4 | 0.0782% | 0.0090% | 0.0711% | 0.0090% |
| | N3 | 0.4045% | 1.5192% | 0.0151% | 0.1022% |
| | P5 | 0.1128% | 0.1867% | 0.0141% | 0.0312% |
| I-09 | P6 | 0.0470% | 1.3248% | 0.0000% | 0.0257% |
| | N4 | 0.0066% | 0.0055% | 0.0044% | 0.0011% |
| | P7 | 0.0176% | 0.0202% | 0.0059% | 0.0014% |
| I-10 | P8 | 0.0135% | 0.0192% | 0.0045% | 0.0011% |
| | N6 | 0.0052% | 0.0000% | 0.0000% | 0.0000% |
| | P10 | 0.0052% | 0.0103% | 0.0026% | 0.0052% |
| I-15 | P11 | 0.0021% | 0.0042% | 0.0000% | 0.0042% |
| | N7 | 0.0031% | 0.0061% | 0.0000% | 0.0031% |
| I-16 | P12 | 0.0000% | 0.0051% | 0.0000% | 0.0026% |
| | P13 | 0.0054% | 0.0065% | 0.0036% | 0.0065% |
| | P14 | 0.0000% | 0.0180% | 0.0000% | 0.0090% |
| I-17 | P16 | 0.0713% | 0.0410% | 0.0713% | 0.0410% |
| I-18 | P17 | 0.0018% | 0.0055% | 0.0018% | 0.0018% |

### Table 4 (Legend)

Results of FIFIC analyses of T-cell response of melanoma patients

and healthy controls to in vitro stimulation with or without

autologous tumour cell lysate.

The first column shows the experiment identification number including those patients (P) and healthy controls (N) shown in column two.

Column three shows the percentage of IFN-γ+ CD69+ cells among CD4+ lymphocytes upon incubation with FM3.29 tumour cell lysate.

Column four shows the percentage of IFN-γ+ CD69+ cells among CD4+ lymphocytes upon incubation without cell lysate.

Column five and six are similar to column three and four, except that data are corrected by removal of events thought to represent “autoflourescent” events*.

Note: *The “autofluorescent” events, which were present in a minority of samples, mimic cells carrying both FITC and PE in approximately equal amounts. They are recognisable by the fact that they form a straight, diagonal line in FITC/PE plots. Similar unwanted signals have been described in the literature (Nomura et al, Cytometry 2000, 40: 60-68).

Examples of FIFIC-plots for a tumour cell lysate-responding patient (P4) and a healthy, non-responding person (N1) is shown in FIG. 4 and FIG. 5, respectively.

FIG. 4 shows a summary of the results of FIFIC analysis of a tumour cell (FM3.29) lysate responding melanoma patient (P4).

FIG. 4a shows the results with no tumour cell lysate added.

FIG. 4b shows the results with tumour cell lysate added.

The response is estimated by 0.08%−0.00917%=0.071%.

FIG. 5 shows a summary of the results of FIFIC analysis of a healthy control (N1) with no response to tumour cell (FM3.29) lysate.

FIG. 5a shows the results with no tumour cell lysate added.

FIG. 5b shows the results with tumour cell lysate added.

Neither with nor without tumour cell lysate added, no IFN-γ+ CD69+ CD4+ lymphocytes are observed, and the response is 0.

Results are graphically displayed in FIG. 6 and FIG. 7. Six out of 14 melanoma patients had a positive T-cell response upon exposure to allogenous melanoma cell lysate, and 3 of these were interpreted as clear positive responders (FIG. 6, group 2-4). In contrast, only 1 out of 6 normal controls showed a slight positive response (FIG. 6, group 1).

FIG. 6 shows a graphical presentation of the specific T-cell response to autologous FM3.29 tumour cell lysate in healthy controls and in melanoma patients grouped according to disease stage.

The y-axis shows response evaluated as % IFN-γ+, CD69+ among CD4+ lymphocytes upon incubation with tumour cell lysate with background subtracted (i.e. spontaneous IFN-γ production from the unstimulated sample).

Each bar on the x-axis represents an individual. Group 1: healthy controls, group 2: stage I melanoma patients, group 3: stage III melanoma patients, and group 4: stage 1V melanoma patients.

FIG. 7 shows a graphical presentation of the T-cell response to FM3.29 tumour cell lysate in melanoma patients according to presence or absence of overt disease at the time of blood test.

The y-axis shows response evaluated as % IFN-γ+, CD69+ among CD4+ lymphocytes upon incubation with tumour cell lysate with background subtracted (i.e. spontaneous IFN-γ production from the unstimulated sample).

Each bar on the x-axis represents an individual. Group 1: melanoma patients with no clinical signs of recidual melanoma, group 2: melanoma patients with clinically detectable overt melanoma disease.

In a study by Letsch et al (Letsch, A et al, Int. J. Cancer 2000, 87: 659-664) using HLA-tissue-type matched, intact autologous tumour cells from several cell cultures, 11 out of 19 patients with metastatic melanoma had a T-cell response with from 0.04% up to 0.81% of peripheral blood mononuclear cells secreting INF-γ (as assessed by ELISPOT). The frequencies of T-cell responses in the responding patients exceeded the mean and 3-fold standard deviation of the T-cell responses observed in 16 healthy individuals. In our study 43% of melanoma patients were responders using lysate from only one tumour cell line. The inclusion of more cell lines would most likely increase the frequency of responders.

Results of our experiments show a high rate of clear positive responders to melanoma-cell lysate in patients with known clinically overt melanoma (2 out of 4 cases) (FIG. 7, group 2), whereas the response in 6 normal controls was absent/negative or very faintly positive (FIG. 7, group 1). Thus, a significant response to the melanoma cell lysate would seem to indicate the presence of malignant melanoma cells in a person.

Additionally, we did not find positive responses to tumour cell lysate in 6 patients previously treated by radical excision of thin melanomas (stage I) (FIG. 6, group 2). These patients have a very high chance of being cured by surgery (approximately 90% 5-year survival-rate (AJCC cancer staging manual 5^{th} ed., American Cancer Society, Lippincot-Raven, Philadelphia 1997)) and most likely had no remaining tumour cells in the body at the time of the blood sample. In contrast, patients which were positively known to have malignant melanoma cells in the body at the time of blood sample due to clinically detectable tumour, often showed a response as did patients with a very high risk of having residual malignancy despite being apparently free from tumour (i.e. patients with stage III and IV disease without clinically detectable remaining tumour) (FIG. 6, group 3-4).

Previous studies suggest that memory T-cells fall into two broad categories on the basis of their activation status (Sallusto, F. et al). Typical memory cells, which may be termed “resting” memory cells, are relatively quiescent and need to be re-activated before expressing effector function. A second category of memory cells displays many of the features of effector cells and may be termed “activated” memory cells. Activated memory cells may represent a subset of cells that retain T-cell receptor contact with small quantities of specific antigen (Sprent, J. and Surh, C. D., Current Opinion in Immunology 2001, 13: 248-254). Thus, activated memory T-cells may be dependent on very recent contact with antigen in vivo, and may, with respect to malignancies, therefore act as an indirect tumour marker. The detection of activated memory T-cells rather than resting memory T-cells may be based on the fact that the first-mentioned T-cells already to some extent are activated and therefore secrete cytokines after only a very brief contact with antigen in vitro (typically in the range of few hours). Resting memory T-cells, in contrast, would require longer antigen contact to be activated (typically many hours to few days).

Thus the existence of and the possibility to detect in vivo antigen-contact-dependent specific T-cells in cancer patients was supported by our data: using short-time T-cell stimulation (incubation of blood with tumour cell lysate for 6 hours), no activated circulating tumour-specific T-cells could be detected in patients who were treated by radical surgery for stage I melanoma 14 days to 41 days prior to the blood test, whereas a high frequency of patients with tumour-specific T-cells was seen in patients with overt tumour or with a high risk of having residual disease at the time of blood test.

As a further consequence, our results suggest that after treatment a high or rising T-cell response to antigen exposure in vitro could indicate residual disease such as occult metastases. Moreover, in patients with no clinically detectable residual disease after treatment, the test may be used in the follow-up as an indicator of recurrence. In this context, a high or rising T-cell response would suggest recurrent disease.

The difference between means of T-cell response for stage I patients and stage III+IV patients was statistically significant (2p=0.045). Thus, our results shows a positive correlation between melanoma disease stage and intensity of T-cell response to melanoma cell lysate (FIG. 6, group 2-4). As stage of disease is a very important prognostic factor (AJCC cancer staging manual 5^{th} ed., American Cancer Society, Lippincot-Raven, Philadelphia 1997) this may likely also be so for the T-cell response test result. Based on the same finding it seems likely also, that a rising response to the test would indicate progressive disease and, thus, poorer prognosis.

As a large number of circulating tumour-specific T-cells would be characteristic for very immunogenic tumours, it would be expected that a high T-cell response prior to therapy would be indicative of an increased chance of an effect of antineoplastic therapy stimulating the immune system. Also, the induction of a response or a rising response induced by such therapy could indicate a higher probability of a positive response to that treatment. In contrast, a declining or unchanged T-cell response would be expected to indicate a poor or declining effect of such therapy. Schmittel et al, for example, showed that in melanoma patients peripheral T-cells against specific tumour antigens could be induced by chemotherapy combined with IFNα +/− IL-2 and that loss of these T-cells was associated with relapse of the disease (Schmittel, A. et al)

### EXAMPLE 15

Incorporation of radioactive Iododeoxyuridine into activated T-cells and imaging of infused labelled cells.

Obtaining T-LAK Cells

C57BL/6J mice, 8-10 weeks of age were obtained from Bomholtgaard (Ry, Denmark). Spleens were removed from C57BL/6J mice and a single-cell suspension of cells was prepared in RPMI-1640. Erythrocytes were lysed with ammonium chloride-potassium buffer at room temperature for 3 min, and the cells were washed twice in RPMI 1640. Subsequently the cells were transferred into plastic flasks (TCC) and cultured at 37°, 5% CO₂ in RPMI 1640 supplemented with 5% heat inactivated FCS and 5% heat inactivated normal human serum (NHS), 10 ml/l 100 x nonessential amino acids (Gibco, Denmark), 50 mM 2-mercaptoethanol (Sigma, St. Luis, USA), 2 mM glutamax, 10 mM Hepes buffer and 20 ml/l (streptomycin 0.8 g/ml and 1.6×10⁵ penicillin), hereafter referred to as LAK medium (LAK-M) at a cell concentration of 2×10⁶ cells/ml. For activation of T cells, the cell suspension was incubated with 0.4 μg/ml of PHA-P (Phytohemagglutinin-P; DIFCO, Detroit Mich.) and 100 Cetus-U/ml of IL-2 (rIL-2) (Chiron corporation, Harefield, U.K.). After 2 days of incubation, non-adherent clusters of activated T cells, were transferred to 50 ml TCC tubes, where they were allowed to sediment for 3-5 minutes, after which the supernatant was gently removed and the cell pellet resuspended in fresh LAK-M containing 100 Cetus-U/ml rIL-2.

For continuous culture, the medium of T-LAK cells was renewed every second day (LAK-M containing 100U/ml IL-2). The cell density was always kept at 0.5-1.5×10⁶ cells/ml.

Labelling of T-LAK Cells with ^{125/124}IUdR.

On the day of labelling 4 days old T-LAK cells were counted and placed in culture at a concentration of 400.000/ml in LAK-M with 100 Cetus-U/ml of IL-2 in the morning.

Approximately 6 hours later ¹²⁵IUdR (0.1 kBq/ml) or ¹²⁴IUdR (1.5 kBq/ml) were added to the culture. The cells were incubated for 18 hours at 37°, 5% CO₂. After labelling the cells were transferred to 50 ml TCC tubes and samples, duplicates, of the cells were taken for gamma counting to evaluate the incorporation efficiency. 200 μl of well-mixed cell suspension was transferred to tubes and 2 ml of LAK-M were added. After centrifugation 10 min 1500 rpm the supernatant was gently transferred to corresponding tubes and 2 ml LAK-M were added to the cells. The samples were counted on a gamma counter.

Cells were washed 3 times in RPMI-1640 with 2% FCS and 100 Cetus-U/ml of IL-2. After the second wash the cells were counted. Cell numbers were counted by microscopic analysis in a Neubauer haemocytometer. Cell viability was determined by trypan blue exclusion test.

In all experiment a culture of control (unlabeled) T-LAK cells corresponding to the labelled cells were included.

Cell Proliferation and Viability of T-LAK Cells After Labelling with ^{125/124}IUdR.

The T-LAK cell proliferation in vitro was assayed by cell counting. After the 3 wash cells were transferred into plastic flasks (TCC) and cultured at 37°, 5% CO₂ in LAK-M with 100 Cetus-U/ml of IL-2 at a cell concentration of 0.25-0.5×10⁶ cells/ml, depending on the time before counting. At respective days cells were transferred to 50 ml TCC tubes and samples were taken for cell counting, viability and gamma counting. Cell numbers were counted by microscopic analysis in a Neubauer haemocytometer. Cell viability was determined by trypan blue exclusion test. Cells were again transferred into plastic flasks (TCC) and cultured at 37°, 5% CO₂ in LAK-M with 100 Cetus-U/ml of IL-2 at a cell concentration of 0.25-0.5×10⁶ cells/ml for 1-2 days and the procedure were repeated. If the labelled cells were used for adoptive transfer the counting was performed on the days corresponding to the days the animals were sacrificed.

In all experiment a culture of control (unlabeled) T-LAK cells corresponding to the labelled cells were included. Results are shown in FIGS. 8 and 9 and demonstrate that the proliferation of lymphocytes labelled with 0.1 kBq/mL or 0.01 kBq/mL ¹²⁵IudR (FIG. 8) corresponds to that of unlabelled control cells and is unimpaired. However, labelling with 1.5 kBq/mL impedes proliferation through increasing the generation time of the labelled cells, while labelling with 15 kBq/mL is toxic to the cells (FIG. 9).

Tumour Cells

B16 (a murine melanoma cell line of C57BL/6 origin, established at the department of Medical Microbiology and Immunology (University of Aarhus, Denmark) was maintained in RPMI-1640 supplemented with 10% heat-inactivated foetal calf serum (FCS), 2 mM glutamax, 10 mM Hepes and antibiotics 20 ml/l (streptomycin 0.8 g/ml and 1.6×10⁵ U/l penicillin), at 37° C. and 5% CO₂. Cells were passages as required to maintain cultures in a log phase growth and adherent cells were detached by exposure to 0.02% EDTA for 4-5 minutes.

Induction of Lung and Subcutaneous Tumours in C57BL/6J mice.

Lung metastases of B16 cells were induced i.v. in C57BL/6 mice, 8-9 weeks of age, by inoculation in the lateral tail vein of 1×10⁶ cells in 0.3 ml RPMI-1640-without NaHCO₃ containing 2% FCS.

For inoculation of subcutaneous tumours in C57B1/6 mice, 8-9 weeks of age were anaesthetised by 3% Halothane/fluothane and shaved on both flanks. Subcutaneous tumours by inoculation s.c in the flanks with 1×10⁵ cells in 0.05-0.1 ml RPMI-1640-without NaHCO₃ containing 2% FCS. Each mouse was injected in both flanks.

Inoculation of T-LAK Cells.

The day prior to inoculation of T-LAK cells potassium iodide was added to the drinking water.

8-10 days after induction of tumours in C57BL/6j mice, 20×10^{6 125/124}IUdR labelled T-LAK cells in a volume of 300 μl of RPMI-1640-without NaHCO₃— with 100 Cetus-U/ml of IL-2, were inoculation i.v. in the lateral tail vein. Intra periotoneal (i.p) injection of 25.000 Cetus U IL-2 in 500 μl PBS-pH 7.4 was performed 4 hours after the initial inoculation and subsequently twice every day.

After respective days mice were sacrificed and transferred to 50 ml TCC tubes and PET scanned.

¹²⁴I PET Scanning of Mice.

The mice were PET scanned using the ECAT EXACT HR whole-body scanner (CTI PET Systems, Knoxville, USA) e.g. in 5% formalin. The mice were scanned individually or in groups of maximum seven. Emission data were acquired over a period of 1-12 hours depending on the number of mice in the batch and the available scan time. Also a 2 min transmission scan was acquired for attenuation correction in order to ensure absolute calibration. The images were reconstructed as 128×128×47 matrices using filtered back projection and a Ramp filter at the Nyquist frequency, resulting in an isotropic spatial resolution (FWHM) of 4 mm. The image quality and detection limit obtainable with ¹²⁴I was validated separately in a phantom study.

Labelling of CTL Cells with ¹²⁴IUdR.

On the day before labelling CTL cells were incubated at a concentration of 500.000/ml in LAK-M with 100 Cetus-U/ml of IL-2.

The day after ¹²⁴IUdR (15 kBq/ml) was added to the culture. The cells were incubated for 24 hours at 37°, 5% CO₂. After labelling the cells were transferred to 50 ml TCC tubes and samples, duplicates, of the cells were taken for gamma counting to evaluate the incorporation efficiency. 200 μl mixed cell suspension were transferred to tubes and 2 ml of LAK-M were added. After centrifugation 10 min 1500 rpm the supernatant was gently transferred to corresponding tubes and 2 ml LAK-M were added to the cells. The samples were counted on a gamma counter.

Cells were washed 3 times in RPMI-1640 with 2% FCS and 100 Cetus-U/ml of IL-2. After the second wash the cells were counted. Cell numbers were counted by microscopic analysis in a Neubauer haemocytometer. Cell viability was determined by trypan blue exclusion test.

Inoculation of CTL Cells.

The day prior to inoculation of CTL cells potassium iodide was added to the drinking water of the mice. 8-10 days after induction of tumours in C57BL/6J mice, 10-20×10^{6 125/124}IUdR labelled CTL cells in a volume of 300μl of RPMI-1640-without NaHCO₃— with 100 Cetus-U/ml of IL-2, were inoculation i.v in the lateral tail vein. Intra periotoneal (i.p) injection of 25.000/50.000 Cetus U IL-2 in 500 μl PBS-pH 7.4 was performed 4 hours after the initial inoculation and hereafter twice every day.

After respective days mice were sacrificed and transferred to 50 ml TCC tubes and PET scanned.

The whole of the disclosure of each document referred to herein is hereby incorporated by reference as if the document were written out here in its entirety.

### REFERENCES

| | |
|---|---|
| 1 | Agace W.W. et al., Curr Opin Cell Biol. 2000. |
| | 12:563-8 |
| 2 | AJCC cancer staging manual 5^{th} ed., American Cancer |
| | Society, Lippincot-Raven, Philadephia 1997 |
| 3 | Andersen M.H. et al Cancer Res. 2001, 61: 869-72 |
| 4 | Altenschmidt, U. et al, J. Immunol. 1997, 159: 5509- |
| | 5515 |
| 5 | Ashton-Rickardt, P.G. and Opferman, J.T. Cell. Mol. |
| | Life Sci. 1999, 56: 69-77 |
| 6 | Baggers, J. et al, J. Clin. Oncol. 2000, 18: 3879- |
| | 3881 |
| 7 | Basse, P.H. APMIS 1995, S55: 5-28 |
| 8 | Beun. G.D. et al, Immunol Today 1994, Jan: |
| | 15 (1): 11-5 |
| 9 | Boczkowski, D. et al, J.Exp. Med. Vol. 184, Aug |
| | 1996, pp 465-472 |
| 10 | Bodnar, A.G. et al Science 1998, 279: 349-352 |
| 11 | Botti, C. et al, Eur. J. Nucl. Med. 1997. May: |
| | 24 (5): 497-504 |
| 12 | Cameron, R.B. et al., J. Exp. Med. 1990, 171: 249- |
| | 263 |
| 13 | Celik M.S. et al, J Colloid Interface Sci. 1998, |
| | 203: 254-9 |
| 14 | Chang, J.W. et al, Anticancer Res. 2000, 20: 1329- |
| | 1336 |
| 15 | Claudio, P.P. et al, Cancer Res. 2001, 61: 462-8 |
| 16 | Coderre, J.A. et al, Radiation Research (1999) 151 |
| | pp 1-18 |
| 17 | Cormier, J.N. et al, Int. J. Cancer 1998, 75: 517- |
| | 524 |
| 18 | Costello, E. et al, Gene Ther. 2000, 7: 596-604 |
| 19 | Davidoff, M. et al, J.Pediatric. Surg. 2001, 36: |
| | 30-36 |
| 20 | Demir, G. et al, Anticancer Res. 1999, 19: 3517- |
| | 3520 |
| 21 | Ding, I. Et al, Cancer Res. 2001, 61: 526-31 |
| 22 | Dodd, et al, Biophysical Jnl. 76, Jan 1999, 103-109 |
| 23 | Dunbar P. R., et al, J. Immunol., 1999, 162: 6959- |
| | 62 |
| 24 | Elsasser-Beile, U. et al, Br. J. Cancer 2000, 83: |
| | 637-641 |
| 25 | Eshhar, Z., Cancer Immunol Immunother (1997) 45: |
| | pp 131-136 |
| 26 | Fisher, B., et al, J.Clin. Oncol. 1989 Feb; |
| | 7 (2): 250-261 |
| 27 | Gluckman, J.C., et al Cytokines, Cellular and |
| | Molecular Therapy 1997/Vol.3, Pg 187-196 |
| 28 | Goldfarb, R.H. et al, In Vivo 2000, 14: 101-104 |
| 29 | Griffith, K.D. et al, J. Natl Cancer Inst. Nov. |
| | 1989 15: 81 (22): 1709-17 |
| 30 | Guan, L., et al, Proc. Natl. Acad. Sci. USA 1998, |
| | Oct 27; 95 (22): 13206-10 |
| 31 | Guenther I. et al, Nuclear Medicine & Biology, Vol. |
| | 25, pp 359-365, 1998 |
| 32 | Gyger, M. et al, Bone Marrow Transplant, 2000, 26: |
| | 1-16 |
| 33 | Haruta, I. Et al, J. Immunotherapy 19 (3): 218-223 |
| 34 | Hawthorne, M.F., Molecular Medicin Today, Apr. |
| | 1998, pp 174-181 |
| 35 | Hirschowitz. E.A. et al, Cancer Gene Ther. 1999, 6: |
| | 491-498 |
| 36 | Hwu. P. and Rosenberg, S.A. Cancer Detection and |
| | Prevention 1994, 18: 43-50 |
| 37 | Jakob. C., et al, FEBS Lett 1995 Jul 10; 368 (1): 5-9 |
| 38 | Josephson L, et al, Bio Conjug Chem 1999. Mar-Apr; |
| | 10(2): 196-91 |
| 39 | Ju, D.W et al, Gene Ther. 2000, 7: 1672-9 |
| 40 | Kammula, U.S. et al, J. Immunol. 1999, 163: 6867- |
| | 6875 |
| 41 | Kim, J.A. Cancer 1999, 86: 22-30 |
| 42 | Kawakami, Y., and Rosenberg, S.A. Immunologic |
| | Research 1997, 16: 313-339 |
| 43 | Korf, J., et al, The Journal of Nuclear Medicine, |
| | Vol. 39, No.5 May 1998. Pgs 886-841 |
| 44 | Laabi, Y. and Strasser, A., Science, 2000, 289: |
| | 883-4 |
| 45 | Lalvani, A., et al, J. Exp. Med. 1997, 186: 859-865 |
| 46 | Lalvani, A., et al, J. Immunological Methods 210 |
| | (1997) pp 65-77 |
| 47 | Letsch, A. et al, Int. J. Cancer 2000, 87: 659-664 |
| 48 | Lewin M. et al Nature BioTechnology Vol.18, April |
| | 2000 pp 410-444 |
| 49 | Lewko, W.M. et al, Cancer Biother. Radiopharm. |
| | 2000, 87: 659-664 |
| 50 | Luxembourg, A.T. et al, Nature Biotechnology Vol.16 |
| | March 1998, pp 281-285 |
| 51 | Mishra, P., et al, Nucl. Med. Commun. 1994 |
| | Sep: 15(9): 723-9 |
| 52 | Mitchell, M.S. et al, Cancer Res. 1988, 48: 5883-93 |
| 53 | Morse, M.A. et al, J. Clin. Oncol. 2000, 18: 3883- |
| | 3893 |
| 54 | Mortarini, R. et al, Cancer Res. 2000, 60: 3559- |
| | 3568 |
| 55 | Mukherji, B. et al J. Med. Biol. Vol 15, No.4 |
| | pp 419-427 1988 |
| 56 | Nestle, F.O. et al, Nat. Med. 1998, 4: 328-32 |
| 57 | Niranjan, A. et al, Mol. Ther. 2000, 2: 114-120 |
| 58 | Nomura, S. et al, Cytometry 2000, 40: 60-68 |
| 59 | Peters, J.H., et al, Immunology Today, Jun. 1996, |
| | Vol.17, No.6, pp 273-278 |
| 60 | Plebanski, M., Eur.J.Immunol. 1994, 25:1783-1787 |
| 61 | Pockaj, B.A., et al, Cancer, Mar. 15, 1994, Vol. |
| | 73, No.6 pp 1731-1737 |
| 62 | Protti, M.P., et al, Cancer Res. 1996, Mar |
| | 15: 56(6): 1210-3 |
| 63 | Romero, P. et al, Mol. Med. Today 1998, 4: 305-312 |
| 64 | Rosenberg, S.A. Annals of Surgery 1993, 218: 455- |
| | 464 |
| 65 | Roth, M.D. et al, Cancer Res. 2000, 60: 1934-1941 |
| 66 | Protti, M.P., et al, Cancer Research, 56: Mar. 15, |
| | 1996 (1210-1213) |
| 67 | Ranadive, G.N. et al, Nucl. Med. Biol. 1993 |
| | Jan;20 (1): 1-6 |
| 68 | Safwat, A. Radiation Res. 2000, 153: 599-604 |
| 69 | Sallusto, F. et al, Nature 1999, 401: 708-71 |
| 70 | Sano, T., Bioconjug Chem. 1999 Sep-Oct;10(5): 905-11 |
| 71 | Sansom, D.M. et al, Immunology 1993, 80: 242-7 |
| 72 | Santin, A.D. et al, Am J. Obstet. Gynecol. 2000, |
| | 183: 601-609 |
| 73 | Santin, A.D. et al, Obstet, Gynecol, 2000, 96: 422- |
| | 430 |
| 74 | Santin, A.D. et al, Eur. J. Gynecol, Oncol. 2000, |
| | 21: 17-23 |
| 75 | Santin, A.D. et al, Gynecol. Oncol. 1996, 60: 468- |
| | 474 |
| 76 | Santin, A.D. et al, Jnl. Of Virology 1999, 73: |
| | 5402-5410 |
| 77 | Santin, A.D. et al, Gynecol. Obstet. Invest. 2000, |
| | 49: 194-203 |
| 78 | Schmidt-Wolf, I.G. et al, Br. J. Cancer 1999, 81: |
| | 1009-1016 |
| 79 | Schmittel, A. et al, J. Immunother. 2000, 23: 289- |
| | 295 |
| 80 | Schmittel, A. et al, Int. J. Cancer 1999, 80: 39-43 |
| 81 | Schmitz, M. et al, Cancer Res. 2000, 60 (17): 4845-9 |
| 82 | Shiloni, E. et al, Cancer Immunol. Immunother. |
| | 1993, 37: 286-292 |
| 83 | Sporn, J.R. et al, Cancer Immunology Immunotherapy, |
| | 1993, pp 175-180 |
| 84 | Sprent, J. and Surh, C.D., Current Opinion in |
| | Immunology 2001, 13: 248-254 |
| 85 | Straten, P., et al, The Journal of immunology, |
| | 1999, 163: 443-447 |
| 86 | Straten, P., et al, Cancer Immunol. Immunother. |
| | 1999, 34: 386-395 |
| 87 | Sussman, M.S. et al, Magn. Reson. Med. 40 (6): 890- |
| | 9, 1998 |
| 88 | Tanaka, F., et al, Cancer Immunology Immunology |
| | (1997) 44, pp 21-26 |
| 89 | Thery, C., et al, J. Cell Biol. 1999 Nov |
| | 1: 147(3): 599-610 |
| 90 | Vella, A.T. et al, Proc. Natl. Acad. Sci. 1998, 95: |
| | 3810-5 |
| 91 | Wang, R.F. J. Mol. Med. 1999, 77: 640-655 |
| 92 | Westermann, J. et al, Cancer Immunol. Immunother. |
| | 2001, 49: 613-20 |
| 93 | Willemsen, R.A. et al, Gene Ther. 2000, 7: 1369- |
| | 1377 |
| 94 | Zitvogel L, Nat. Med. 1998, May; 4(5): 594-600 |

1. A method of selectively activating or proliferating one or more T-cell clones each specific for an antigen associated with a pathological process, comprising culturing, under T-cell activating or proliferative conditions, a T-cell mixture potentially including cells having a memory specific for at least one said antigen with an effective antigen presenting agent and an antigen mixture, said conditions being sufficiently selective that substantially only T-cells already primed to recognise said antigens are caused to become activated or to proliferate, wherein said antigen mixture has been derived from a microorganism or cell associated with said pathological process by a process comprising lysis, extraction of protein or peptide mixtures, or by the formation of apoptopic bodies, or by being produced in situ from mRNA or DNA derived from said cell or a pathogenic microorganism associated with said pathological process.

2. A method as claimed in claim 1, wherein in preparing said antigen mixture, after cell lysis, cell membrane debris is removed.

3. A method as claimed in claim 1 or claim 2, wherein in preparing the antigen mixture, immune stimulating agents are added or their concentration in the mixture is boosted or immune suppressing agents are removed or blocked.

4. A method as claimed in claim 1, wherein the cell from which said antigen mixture is derived is allogeneic with respect to said T-cells.

5. A method as claimed in any preceding claim, wherein said antigen presenting agent comprises antigen presenting cells.

6. A method as claimed in claim 5, wherein said antigen presenting cells are at least predominantly dendritic cells.

7. A method as claimed in claim 5, wherein said antigen presenting cells are at least predominantly monocytes.

8. A method as claimed in claim 5, wherein said antigen presenting cells are derived from peripheral blood leukocytes.

9. A method as claimed in any one of claims 1 to 4, wherein said antigen presenting agent comprises exosomes.

10. A method as claimed in any preceding claim, wherein the T-cells are PBL (Peripheral Blood Leukocytes) or lymphocytes obtained from fluid in body cavities, the lymphatic system, bone marrow or cerebrospinal fluid.

11. A method as claimed in any preceding claim, wherein said activated T-cells are selectively extracted using antibody coated magnetic beads.

12. A method as claimed in any preceding claim, wherein said antigen mixture derives from cancer cells or a cancer cell line.

13. A method as claimed in any one of claims 1 to 11, wherein said antigen mixture derives from a site of pathology infected by a parasite, fungus, bacterium, virus, or prion or from a parasite, fungus, bacterium, virus or prion.

14. A method as claimed in any one of claims 1 to 11, wherein said antigen mixture is derived from a site of pathology giving rise to an allergic, autoimmune, chronic inflammatory or granulomatous disease or a disease wherein abnormal proteins or other compounds are deposited in tissue.

15. A method for detecting prior exposure of an individual mammal's immune system to an antigen associated with a pathological process, comprising obtaining a sample from a mammal, said sample containing T-cells, exposing said T-cells to a library of antigens forming a complex antigen mixture, and detecting a pre-existing T-cell specificity for an unknown antigen in said complex antigen mixture.

16. A method as claimed in claim 15, wherein said detection of specificity comprises attempting to selectively activate or proliferate one or more T-cell clones each specific for an antigen associated with a pathological process, comprising culturing, under T-cell activating or proliferative conditions, a T-cell mixture from said sample, potentially including T-cells having a preexisting specificity for at least one said antigen, with an effective antigen presenting agent and a said antigen mixture, said antigen mixture being derived from a microorganism or cell of a type associated with said pathological process by a process comprising lysis, extraction of proteins or a peptide mixture, or by the formation of apoptopic bodies, or being produced in situ from mRNA or DNA derived from said cell or pathogenic microorganism associated with said pathological process.

17. A method as claimed in claim 15, comprising exposing said T-cells to a capture agent comprising said antigens of said library so as to bind to said capture agent T-cells having a pre-existing specificity for an antigen in said library.

18. A method as claimed in claim 17, wherein said library of antigens comprises peptides bound to MHC molecules.

19. A method for detecting prior exposure of an individual mammal's immune system to an antigen associated with a pathological process, comprising obtaining a sample from a mammal, said sample containing T-cells, attempting to selectively activate or proliferate one or more T-cell clones each specific for an antigen associated with a pathological process, comprising culturing, under T-cell activating or proliferative conditions, a T-cell mixture from said sample, potentially including T-cells having a pre-existing specificity for at least one said antigen, with an effective antigen presenting agent and an antigen mixture, said antigen mixture being derived from a microorganism or cell of a type associated with said pathological process by a process comprising lysis or by the formation of apoptopic bodies, or being produced in situ from mRNA or DNA derived from said cell or pathogenic microorganism associated with said pathological process.

20. A method as claimed in any one of claims 15 to 19, wherein said mammal is substantially asymptomatic with respect to said pathogenic process.

21. A method as claimed in any one of claims 15 to 20, wherein the antigens in said antigen mixture are unknown.

22. A method as claimed in any one of claims 15 to 21, wherein said sample contains cells representative in antigen recognition capabilities of the whole memory cell population of said mammal, including T-cells, B-cells, NK-cells and monocytes.

23. A method as claimed in any one of claims 15 to 22, wherein said antigen mixture is derived from multiple cell types associated with respective pathological processes.

24. A method as claimed in any one of claims 15 to 23, wherein said exposure of T-cells to antigens is repeated using one or more further antigen mixtures each being derived from one or more cell types associated with a or a respective pathological process.

25. A method as claimed in any one of claims 15 to 24, wherein the cell from which said antigen mixture is derived is allogeneic with respect to said T-cells.

26. A method as claimed in claim 19 or any one of claims 20 to 25 when directly or indirectly dependent on claim 19, wherein said antigen presenting agent is at least predominantly dendritic cells.

27. A method as claimed in claim 19 or any one of claims 20 to 25 when directly or indirectly dependent on claim 19, wherein said antigen presenting agent is at least predominantly monocytes.

28. A method as claimed in claim 19 or any one of claims 20 to 25 when directly or indirectly dependent on claim 19, wherein said antigen presenting agent comprises exosomes.

29. A method as claimed in any one of claims 15 to 28, wherein the T-cells are PBL (peripheral blood leukocytes) or lymphocytes obtained from fluid in body cavities, the lymphatic system, bone marrow or cerebrospinal fluid.

30. A method as claimed in any one of claims 15 to 29, wherein said antigen mixture derives from a cancer cell or a cancer cell line.

31. A method as claimed in claim 30, wherein said exposure of T-cells is conducted against a panel of antigen mixtures derived from respective cancer cell types.

32. A method as claimed in any one of claims 15 to 29, wherein said antigen mixture derives from a cell infected by a parasite, fungus, bacterium, virus, or prion or from a parasite, fungus, bacterium, virus, or prion.

33. A method of producing labelled T-cells adapted to migrate to the location of an antigen producing microorganism, cell or cell cluster in a mammal or other localised process generated in response to a pathogen, comprising conjugating a detectable label to T-cells which have been purified or selectively multiplied to have a specificity for an antigen produced by said antigen producing microorganism, cell, cell cluster or process.

34. A method as claimed in claim 33, comprising selectively multiplying in culture by a method as claimed in claim 1, T-cells from said mammal specific for an antigen produced by said antigen producing microorganism, cell, cell cluster or process, and conjugating a detectable label to said T-cells.

35. A method as claimed in claim 33, wherein T-cells are purified using magnetic beads coated with antigen presenting molecules presenting peptides extracted from an antigen producing cell, cell cluster or microorganism in a mammal or other localised process.

36. A method as claimed in any one of claims 33 to 35, wherein said label is a radio-label, an X-ray contrast label, an MR contrast label, or a fluorescent label.

37. A method as claimed in claim 36, wherein said label is a Ferrous MR-contrast agent or a Gd-MR-contrast agent.

38. A method as claimed in claim 36, wherein said label is ¹¹¹In, ⁹⁹Tc, ⁵⁵Cr, ⁵⁷Cr, ¹¹⁰In, ⁸⁶Y, ⁷⁶Br, ¹²⁴I, ¹⁸F, ⁵⁵Co, ⁵²Fe, ⁶⁶Ga, ⁵²Mn, ⁴⁸V, ⁸⁴Rb, ⁵⁶Co, ⁵⁸Co, ⁵¹Cr or ¹²³I.

39. A method of determining the location of an antigen producing microorganism, cell or cell cluster in a mammal, or other localised process generated in labelled T-cells produced according to any one of claims 33 to 38, to the mammal that was the original source of said T-cells, allowing said T-cells to migrate to the location of said antigen producing microorganism, cell or cell cluster or localised process, and detecting the location of said migrated T-cells from said label.

40. T-cells specific for an antigen, said T-cells being conjugated to a cytotoxic material, or to a material capable of being transformed in vivo into a cytotoxic material, or capable of causing a pro-form of a cytotoxic material to be transformed in the local vicinity of the T-cell into said cytotoxic material, or to a material capable of enhancing damage caused by applied radiation.

41. T-cells as claimed in claim 40, which are conjugated to a potentially cytotoxic material capable of being transformed into cytotoxic form in the body of a mammal by localised administration of a stimulus to the body of the mammal.

42. T-cells as claimed in claim 41, wherein the potentially cytotoxic material is an isotope which is transformable in vivo by bombardment with thermal neutrons to produce α particles or larger high energy particles.

43. T-cells as claimed in claim 42, wherein the isotope is ¹⁰B.

44. T-cells as claimed in claim 42 or claim 43, having ingested therein nanobeads comprising said isotope.

45. T-cells as claimed in claim 40, wherein said cytotoxic material is a radionuclide.

46. T-cells as claimed in claim 45, wherein said radionuclide is incorporated in nanobeads ingested by said T-cells.

47. Nanobeads comprising a core having a polymer coating incorporating a membrane translocation signal peptide (MTSP) and comprising a neutron capture isotope transformable upon neutron bombardment to produce a particles or larger high energy fragments or comprising a radionuclide.

48. A method as claimed in any one of claims 15 to 32, further comprising measuring the numbers of T-cells having a pre-existing specificity or the extent of selective T-cell activation or proliferation obtained and estimating therefrom the extent of current pathology, the likelihood of successful treatment, the progress of treatment, the recurrence of the pathology, the prognosis with respect to recurrence or final outcome of the pathology.

49. A method as claimed in any one of claims 15 to 32, further comprising attempting selective further activation or proliferation of T-cells specific for an unknown antigen using a known or unknown antigen or a mixture of known or unknown antigens.

50. A method as claimed in any one of claims 15 to 32, further comprising determining the identity of an antigen to which said T-cells have specificity by attempting to produce selective further activation or proliferation of said T-cells using a suspected antigen and determining whether said further activation or proliferation is produced in an antigen dependent manner.

51. A method as claimed in claim 49 or claim 50, wherein said antigen activated or proliferated T-cells are selectively extracted prior to further activation or proliferation.
